SEQUENCE LISTING (15 sequences)

SEQ ID NO: 1 - DNA, 6872 bp, Artificial
  plasmid
ttattggacactagataatcatcacatgttaccacaaaattatataatgtataaatgcgaaattattaaacgcaaatatccatgggaaaacgcgcagtatacagacgattttttacagtatttggagagttttataggaagtatatagagtagaaccagaattttgtaaaaataaatcacatttttatactaatatgaaacaactatcgatagttatattgctactatcgatagtatatacaaccaaacctcatcctacacagatatcaaaaaaactaggcgatgatgctactctatcgtgtaatagaaacaatacacatggatatcttgtcatgagttcttggtataagaaaccagactccattattctcttagcagccaaaaacgatgtcgtatactttgatgattatacagcggataaagtatcatacgattcaccgtatgatactctagctacaattattacaattaaatcattgacatctggagatgcaggtacttatatatgcgcattctttataacatcaacaaatgatacggataaaatagattatgaagaattcgtcgacgcggccgcctgcagaagcttggtaccatttatagcatagaaaaaaacaaaatgaaattctactatatttttacatacatatattctaaatatgaaagtggtgattgtgactagcgtagcatcgcttctagacatctatatactatatagtaataccaatactcaagactacgaaactgatacaatctcttatcatgtgggtaatgttctcgatgtcgatagccatatgcccggtagttgcgatatacataaactgatcactaattccaaacccacccgctttttatagtaagtttttcacccataaataataaatacaataattaatttctcgtaaaagtagaaaatatattctaatttattgcacggtaaggaagtagaatcataaagaacagtgacatggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctcaaggtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatcaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccggtcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccgttcgctaccattaccagttggtctggtgtcaaaaataaggatcctcgataccaacaacggtagaaagtgttacaatatctactacaaaatatacaactagtgactttatagagatatttggcattgtttcactaattttattattggccgtggcgattttctgtattatatattatttctgtagtggacggtctcgtaaacaagaaacaaatatattatagattttaactcagataaatgtctggaataattaaatctatcgttttgagcggaccatctggttccggcaagacagctatagtcaggagactcttacaagattatggaaatatatttggatttgtggtatcccataccactagatttcctcgtcctatggaacgagaaggtgtcgtctaccattacgttaacagagaggccatttggaagggaatagccgctggaaacttgctagaacatacagagtttttgggaaatatttatgggacttctaaaacatccatgaacacagctgctattaataatcgtatatgtgttatggatttaaacattgacggagttaggagtcttaaaaacacatacttgatgccttactctgtttatataagacctacatctcttaaaatggtagaaactgcatgccctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagagctc

SEQ ID NO: 2 - DNA, 5508 bp, Artificial
  plasmid
gagctcataaatagtaaaccgatagtgtataaagattgtgcaatgcttttgcgatcaataaatggatcacaaccagtatctgttgacgatgttcttcgcagatgatgattcattttttaaatatttagctagtcaagacgatgaatcttcattatccgatatattgcaaattacccaatatcttgattttttattgttgttattgatacagtcaaaaaataaactagaggctgtaggtcattgttatgaatctctttcggaggaatatagacaattggcgaaatttacagacactcaagagtttaaaaaactatttaataaggttcctattgctacagacggacgcgttaaacttaataaagggtatttatttgactttgttgtcagtatgatgagatttaaaaaagagtcatctataataccaaacatagatccggttcgatacatagatcctcgtcgagatatagtattttctaacgtaatggatatattaaagtctaataaagtgaacaataattaaatttttattgtcacctaggaattcgtcgacgcggccgcctgcagaagcttggtaccatttatattccaaaaaaaaaaaataaaatttcaatttttacatacatatattctaaatatgaaagtggtgattgtgactagcgtagcatcgcttctagacatctatatactatatagtaataccaatactcaagactacgaaactgatacaatctcttatcatgtgggtaatgttctcgatgtcgatagccatatgcccggtagttgcgatatacataaactgatcactaattccaaacccacccgctttttatagtaagtttttcacccataaataataaatacaataattaatttctcgtaaaagtagaaaatatattctaatttattgcacggtaaggaagtagaatcataaagaacagtgacatggatccggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggcttttcgctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaggatccggaaatggtagtaaaactgacagcggtatgtatgaaatgctttaaagaggcgtcgttttctaaacgtttgggaacggaaaccgagatcgaaataattggtggtgaagatatgtatcaatccgtatgcagaaagtgttacatcaacgaatgataatttttctataaaaaactaaaaataaacattgattaaattttaatataatacttaaaaatggatgttgtgtcactggataaaccgtttatgtattttgaggaaatagataatgaactagaatacgaaccagaaagtgcaaatgaggttgctaaaaaacttccatatcagggacaattaaaactattactaggagaattgttttttcttagtaagctacagagacacggtatattggacggtgccactgtagtgtatataggatcggctcctggtacacacatacgttatctacgtgatcatttctataatttaggggtaatcatcaaatggatgctaattgacgggcatgccctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 3 - DNA, 6820 bp, Artificial
  plasmid
ttattggacactagataatcatcacatgttaccacaaaattatataatgtataaatgcgaaattattaaacgcaaatatccatgggaaaacgcgcagtatacagacgattttttacagtatttggagagttttataggaagtatatagagtagaaccagaattttgtaaaaataaatcacatttttatactaatatgaaacaactatcgatagttatattgctactatcgatagtatatacaaccaaacctcatcctacacagatatcaaaaaaactaggcgatgatgctactctatcgtgtaatagaaacaatacacatggatatcttgtcatgagttcttggtataagaaaccagactccattattctcttagcagccaaaaacgatgtcgtatactttgatgattatacagcggataaagtatcatacgattcaccgtatgatactctagctacaattattacaattaaatcattgacatctggagatgcaggtacttatatatgcgcattctttataacatcaacaaatgatacggataaaatagattatgaagaattcgtcgacgcggccgcctgcagaagcttggtaccatgggtatttatattccaaaaaaaaaaaataaaatttcaatttttgctctagacatctatatactatatagtaataccaatactcaagactacgaaactgatacaatctcttatcatgtgggtaatgttctcgatgtcgatagccatatgcccggtagttgcgatatacataaactgatcactaattccaaacccacccgctttttatagtaagtttttcacccataaataataaatacaataattaatttctcgtaaaagtagaaaatatattctaatttattgcacggtaaggaagtagaatcataaagaacagtgacatggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctcaaggtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatcaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccggtcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccgttcgctaccattaccagttggtctggtgtcaaaaataaggatcctcgataccaacaacggtagaaagtgttacaatatctactacaaaatatacaactagtgactttatagagatatttggcattgtttcactaattttattattggccgtggcgattttctgtattatatattatttctgtagtggacggtctcgtaaacaagaaacaaatatattatagattttaactcagataaatgtctggaataattaaatctatcgttttgagcggaccatctggttccggcaagacagctatagtcaggagactcttacaagattatggaaatatatttggatttgtggtatcccataccactagatttcctcgtcctatggaacgagaaggtgtcgtctaccattacgttaacagagaggccatttggaagggaatagccgctggaaacttgctagaacatacagagtttttgggaaatatttatgggacttctaaaacatccatgaacacagctgctattaataatcgtatatgtgttatggatttaaacattgacggagttaggagtcttaaaaacacatacttgatgccttactctgtttatataagacctacatctcttaaaatggtagaaactgcatgccctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagagctc

SEQ ID NO: 4 - DNA, 8373 bp, Artificial
  plasmid
ttattggacactagataatcatcacatgttaccacaaaattatataatgtataaatgcgaaattattaaacgcaaatatccatgggaaaacgcgcagtatacagacgattttttacagtatttggagagttttataggaagtatatagagtagaaccagaattttgtaaaaataaatcacatttttatactaatatgaaacaactatcgatagttatattgctactatcgatagtatatacaaccaaacctcatcctacacagatatcaaaaaaactaggcgatgatgctactctatcgtgtaatagaaacaatacacatggatatcttgtcatgagttcttggtataagaaaccagactccattattctcttagcagccaaaaacgatgtcgtatactttgatgattatacagcggataaagtatcatacgattcaccgtatgatactctagctacaattattacaattaaatcattgacatctggagatgcaggtacttatatatgcgcattctttataacatcaacaaatgatacggataaaatagattatgaagaattcgtcgactcacagtccggtctcacccccgctcttgtatgattcccatgaagatatgatcttcccgctttggggagtgactgacacctccctccctgtccctctgagattgtgttgtgtaggttccgatcgattgactcttctccagcatgtcatcaggaaaattatcaacatcaaggcagtcagggcccctgcactcagtaatacatacttcccccagttcgggagacccaagtcaactcctgagatccgttcgtgcacatcgggaaggtgaacttcaacaaaatcctcagcctcgtcaccgttcttgaaaacggtagacgggtctgccagggggtgcataagggggataaccgaggataccaacaactccatatgttgctggaggagggatgattgcatctctgggattaagacattgccgtcaggtcctaatattataccattgaaaaataccccgtttacatgaggatgacacctccccccaactcttaaacacccttttgaagggatgatctcattccaagttctgactgacttgtagtgagcatcggcttccatcaaggtcttgttgaatatggtatatgcttttccaaacccagggacaagttttcttaaatgactgagacgtctgaaactcactgacttggtggtcatgatggactctagtgcatccagacactcctctctcttcttgaccaactcctctacaacaaggtgctcaatttcgtctgagcgaaagtcgtgcaaattgatcaactgaccgggagggcaccatttggtttcatttgatgtttgcatcgagacccatgttccatccataagtctaagtccgagaactccacataactggagtttgcatgctccttttaaagacttatataggcctctttcatctacaaagccgcaagtctcactccctttggatgctctcttccctctactattggtaaaaatgtcacaagacatccctagtctcggattctcgggcatccaaatggtgtaatcgtggttagtggagcagtaggtagaagacaccgctactcctgagcacttcccgccagggaagaccctcgagtgaagggatctgtcatatgggtccaaatctgccacacttggagatatgataacgagagactccttggtggtttttacagttcgaagccagtggtagtcagggtacggattgtgtagagactcttcatatctggggtcaccggccatcttccagttgtacgcggctctacatgcatctggtgttgggcggaaatgctttcttttgaacgtggttgtgacataaccaacgaagttagtgtaggtttcagcctccgtcacaacgcctgtgcaagtgaacccgttcatttttatggctgagatgtatccaactttaagttccatgtaggagaaccctgacaggttggtgcatccttcgtcctccactaccaaattgtttgggcagctgaggtgatgtatgtcaatcgggctccagggaccaagcttgtctggtatcgtgtaaatagggaatttcccaaaacacaatggaaaaaccagaaggggtacaaacaggagagcctgaggaaccggtaccatgggtatttatattccaaaaaaaaaaaataaaatttcaatttttgctctagacatctatatactatatagtaataccaatactcaagactacgaaactgatacaatctcttatcatgtgggtaatgttctcgatgtcgatagccatatgcccggtagttgcgatatacataaactgatcactaattccaaacccacccgctttttatagtaagtttttcacccataaataataaatacaataattaatttctcgtaaaagtagaaaatatattctaatttattgcacggtaaggaagtagaatcataaagaacagtgacatggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctcaaggtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatcaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccggtcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccgttcgctaccattaccagttggtctggtgtcaaaaataaggatcctcgataccaacaacggtagaaagtgttacaatatctactacaaaatatacaactagtgactttatagagatatttggcattgtttcactaattttattattggccgtggcgattttctgtattatatattatttctgtagtggacggtctcgtaaacaagaaacaaatatattatagattttaactcagataaatgtctggaataattaaatctatcgttttgagcggaccatctggttccggcaagacagctatagtcaggagactcttacaagattatggaaatatatttggatttgtggtatcccataccactagatttcctcgtcctatggaacgagaaggtgtcgtctaccattacgttaacagagaggccatttggaagggaatagccgctggaaacttgctagaacatacagagtttttgggaaatatttatgggacttctaaaacatccatgaacacagctgctattaataatcgtatatgtgttatggatttaaacattgacggagttaggagtcttaaaaacacatacttgatgccttactctgtttatataagacctacatctcttaaaatggtagaaactgcatgccctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagagctc

SEQ ID NO: 5 - DNA, 1179 bp, Chlamydophila felis
  misc_feature (1)..(1179): MOMP gene for major outer membrane protein
atgaaaaaactcttaaaatcggcattattatttgccgctgcgggttccgctctctccttacaagccttgcctgtagggaatccagctgaaccaagtttattaatcgatggcactatgtgggaaggtgcctcaggagatccttgtgatccttgtgctacttggtgtgatgctatcagcatccgtgcaggattctacggagattatgttttcgatcgtatattaaaagttgatgttaataaaaccatcagcggaatggctgcggctccaacagcagcttctggaactgcaagcaacaccactgtcgctgccgacagatcaaattttgcctacggcaaacatcttcaagatgccgaatggtgcaccaatgctgcttacttagcattaaatatttgggatcgttttgatgttttctgcacgctaggagcgtctaatggttacttcaaagcaagttctgatgcatttaaccttgtcggattgattggtcttgcaggaactgatttcgccaatcagcgtccaaacgttgaaatttctcaaggcattgtagagctatacacagataccgcattttcttggagcgttggtgctcgcggagctttgtgggaatgtggttgtgcaactttgggagctgaattccaatatgctcaatccaatcctaaaattgaaatgctcaatgtaacctctagcccagcacaattcatgatacacaagcctagaggatacaaagggactgcagcaaacttccccttacctgtagcagctggcacagcaactgcaacagatactaaatcagctactgttaagtaccatgaatggcaagtaggattggctctttcatacagattgaacatgcttgttccatacattggggtaaattggtcaagagctactttcgatgctgacactatccgcattgctcaacctaaattggcctcagcaatcctaaacttaacaacctggaacccaactcttttaggagtggccacaactttagacacctccaacaaatatgctgacttcatgcaaatcgtttctatgcaaatcaacaagatgaagtctagaaaagcttgtggtattgctgttggagcaactttaatcgacgctgataaatggtccattactggtgaagcacgcttaatcgacgaaagagctgctcacattaatgctcaattcagattctaa

SEQ ID NO: 6 - DNA, 1635 bp, Feline calicivirus
  misc_feature (1)..(1635): Capsid protein from strain 255 with 372 base pair N-terminal deletion and BamHI site mutation
gctgatgatgggtctatcacgtcacctgagcagggaaccatggttggtggtgtgattgcagagcctagtgcccaaatgtcaactgcagctgatatggccacaggaaagagtgtcgactctgaatgggaggcattcttctcattccataccagtgtcaattggagtacatctgaagcacaaggcaagattcttttcaagcaatctttaggaccactacttaacccataccttgagcacctagcaaaactctatgttgcttggtcgggatctgttgatgttagattttctatttctggatctggtgtctttggaggtaagttggctgccattgttgtgcctccaggggtcaaccctgtacaaagcacatcaatgctccaatacccccacgttctctttgatgctcgtcaagtggaacctgttatcttctcaattcctgatctaaggagcactctgtaccaccttatgtctgacattgataccacatccctcgtaatcatggtgtacaatgatcttatcaatccgtatgctagtgattctaattcttctggatgtattgtcactgttgaaactaagcctggaccagatttcaagttccatctactaaaaccccccggttccatgctaactcacggctccgtgccgtctgatttgatcccaaagtcctcttccctctggattggcaatcggcactggaccgatataactgactttgtaattcggccatttgtattccaagccaaccgtcacttcgactttaatcaggagacggctggttggagcacgccaagattccgaccaatcacaataacaattagtgagaaggatggctccaaattgggaattggggttgcaatggactctatcgttcctggaataccggatggatggccagatactaccatacctgagaagttagtccctgctggcaactatgcaatcgccaatgggactggaaatgacattactacagccaaagattatgattcggccactgtaattcaaaacaataccaacttcaaaggtatgtatatccgtggatctttacagagagcctggggtgataagaaaatatcaaacaccgcatttattactactgcaaccaggagtgacaacacaattacaccatccaatgtgatagaccccaccaagattgctgtgtaccaggacacccatgtgggcgcggaagtgcaaacatctgacgacactttggccatccttggttacacaggaattggagaggaagcgattggagctgacagggacagggtcgtgcgcatcagtgtactgccagaaactggggctcgcggtggcaaccatcccatcttttacaagaactctattaaactaggttatgtgattagatctatagatgtgttcaactctcaaatcctgcacacatctcgacaactatccctcaataactatcttctcccacctgattctttcgctgtgtaccgaataactgattctaatggttcatggtttgacataggaattgatagtgatggcttctcttttgtcggtgcctccaacgttggtaaattggagtttcctcttactgcctcctacatgggaattcaattggcaaagattcggcttgcctcaaacattaggagttcattgactaaattatga

SEQ ID NO: 7 - DNA, 1635 bp, Feline calicivirus
  misc_feature (1)..(1635): Capsid gene from strain 2280 with 372 base pair N terminal deletion
gctgatgatggttctatcacggcccctgagcaaggaacggttgttggtggggtcattgccgagcctagtgcacaaatgtcaacagctgctgatatggccacagggaaaagcgttgactctgagtgggaggcattcttttccttccacaccagcgtcaactggagtaccacagaaactcaaggaaagattttattcaaacaatctttgggacccctcctaaacccataccttgaacatcttgctaagctgtatgttgcttggtctggatctattgatgttaggttctctatctctggttctggagtatatgggggaaaacttgctgccattgtcgtaccacctggtgtagaccccgttcaaagtacatcaatgctgcaataccctcatgttctctttgacgctcgtcaagtggaaccagttatcttctctattcctgatttaaggagtactctctatcaccttatatctgatactgatactacttcccttgtgattatggtgtataatgatctcattaacccttatgctagtgatacaaactcttctggatgcattgttacagttgaaaccaagccggggccagatttcaagttccaccttctaaaaccacctggatcaatgctgacacacggttcaatacctgctgacctcatcccaaagtcgtcctccctttggattggcaatcgctattggtctgatatcactgaatttgttgtccgtccctttgtcttccaagcaaaccgacactttgattttaatcaggaaactgctgggtggagcacgccgagattccggccaataactgttacagttagtgaaagtggtgggtcaaagcttgggataggtgttgcaactgactacattgttcccggtattccagatggctggccagacacaacaattcctgaaaagcttacccctgcaggtaattatgcaattacaaccagcaataacagtgacattgctacggctactgaatacgaccatgctgatgaaatcaaaaacaacacaaactttaaaagtatgtacatctgtggatcattgcaaagagcttggggtgacaagaagatatctaatactgcttttatcaccacagcagtcaaggaaggtaacagcatcacaccgtctaacacaattgacatgactaagcttgttgtgtaccaggatgctcacgtgggcaatgatgtgcaaacttccgatgtcacccttgcacttcttggttacacaggaattggtgaacaagcaattggttcagatagagatagagtggtgcgaatcagtgtcctaccagaaactggtgcccgtggcggcaaccaccccatcttctacaaaaatacaattaaattgggctatgtgattaggtctattgatgtgtttaactcccagatcctccacacgtccagacaactatccctaaatcactacctgcttccacctgattcctttgctgtctatagaataattgattctaatggttcatggtttgacattggtattgatagtgatggtttctcttttgttggtgtttctagtttacccacactggaatttcctctctctgcctcctacatgggaattcaattggcaaaaatcaggcttgcctcaaatattaggagtagtatgacaaaattatga

SEQ ID NO: 8 - DNA, 1635 bp, Feline calicivirus
  misc_feature (1)..(1635): Strain DD1 capsid gene with N terminal deletion
gctgatgatggctcaatcacaactcctgagcaaggtacaatggtcggcggtgtaattgctgaacccagttctcaaatgtcagctgctgctgatatggctactgggaaaagtgtggactcggaatgggaagccttcttctcatttcacactagtgtcaactggagcacttctgagacacaaggaaagattctctttaaacaatctttaggaccactgcttaacccctacctttcacatcttgcaaagctatatgttgcttggtctggatctatcgaggttagattttctatttcagggtctggtgtttttggagggaaactggctgccattgtcgtgccgccagggatcgatcccgtccaaagcacctccatgctacaataccctcatgtcctcttcgatgctcgtcaggtagaacctgtcatcttctctatccctgatctaagaagcactctctatcattttatgtctgacactgacactacttcccttgcaatcatggtttataatgatctcattaacccttatgctaatgattcaaattcttcgggatgcattgttacggtagaaaccaaaccaggtcctgactttaagttccatttgttgaaacctcccggctcaatgttgactcatggttcagtaccatctgacctgattccgaggtcatcttcatactggactggaaatcggcattggaccgacatcaccgggtttgtaattcgaccttttgtgttccaagccaacagacactttgatttcaatcaggaaactgccggctggagttcaccgagatttcgcccaatttcaatcaatatcagtgttgaaaaagccgcaaaacttggaactggagttgctactgattacattgtccctggcataccagatggttggcctgacaccacaatccctgagaagctgacacctgctggcgattacgccatcgtagatggatcaggcaatgacatcacaactaaggataaatatgaaagtgctgatgtgatcaagaataacaccaatttcaggggcatgtacatttgtggctcacttcaaagagcatggggtgataagaaaatctcaaacactgctttcattactactggaactgttaaggataattcaataatacccagcaataccatagatcaaacaaagatcacagttttccaagacactcacgtcggccatgatcctcaaacctctgatgacacactcgccctactcggttacactgggattggagaagaggcaattggcgccgatcgcgacagggtagttcggatcagtgtccttcctgaaactggtgcgcgtggtggcaatcatcccattttctatagaaactctattaagcttggttatgttctcaaggatattgatgtattcaattctcagattctgcatacctctaagcaactctctctcaatcattacttgctatcacctgattcctttgcagtgtatagaatcactgactcaaatggttcctggtttgatattggcattgataatgatggtttttcttttgttggtgtctcctatattggcaatttggagtttcccctaacagcctcctacatgggaattcaattggcaaaaattcggcttgcctcaaacattaggagttcattgactaaattatga

SEQ ID NO: 9 - DNA, 669 bp, Felis catus
  misc_feature (1)..(669): Feline IL-12 p35 gene
atgtgcccgccgcgtggcctcctccttgtaaccatcctggtcctgttaaaccacctggaccacctcagtttggccaggaacctccccacacccacaccaagcccaggaatgttccagtgcctcaaccactcccaaaccctgctgcgagccatcagcaacacgcttcagaaggctagacaaactctagaattttactcctgcacttccgaagagattgatcatgaagatatcacaaaagataaaaccagcacagtggaggcctgcttaccactggaattaaccatgaatgagagttgcctggcttccagagagatctctctgataactaatgggagttgcctggcctccagaaagacctcttttatgacgaccctgtgccttagcagtatctatgaggacttgaagatgtaccaggtggagttcaaggccatgaatgcaaagctgttaatggatcctaaaaggcagatctttctggatcaaaacatgctgacagctattgatgagctgttacaggccctgaatgtcaacagtgtgactgtgccacagaactcctccttggaagaaccggatttttataaaactaaaatcaagctctgcatacttcttcatgctttcagaattcgtgcagtgaccatcaatagaatgatgagctatctgaattcttcctaa

SEQ ID NO: 10 - DNA, 990 bp, Felis catus
  misc_feature (1)..(990): Feline IL-12 p40 gene
atgcatcctcagcagctggtcatcgcctggttttccctggttttgctggcacctcccctcatggccatatgggaactggagaaaaacgtttatgttgtagagttggactggcaccctgatgcccccggagaaatggtggtcctcacctgcaatactcctgaagaagatgacatcacctggacctctgaccagagcagtgaggtcctaggctctggtaaaactctgaccatccaagtcaaagaatttgcagatgctggccagtatacctgtcataaaggaggcgaggttctgagccattcgttcctcctgatacacaaaaaggaagatggaatttggtccactgatatcttaagggaacagaaagaacccaaaaataagatctttctaaaatgtgaggcaaagaattattctggacgtttcacctgctggtggctgacggcaatcagtaccgatttgaaattcactgtcaaaagcagcagaggctcctctgacccccaaggggtgacttgtggagcagcgacactctcagcagagaaggtcagagtggacaacagggattataagaagtacacagtggagtgtcaggagggcagtgcctgcccggctgccgaggagagcctacccattgaagtcgtggtggacgctattcacaagctcaagtacgaaaactacaccagcagcttcttcatcagggacatcatcaaaccggacccacccaagaacctgcaactgaagccattaaaaaattctcggcatgtggaagtgagctgggaataccctgacacctggagcaccccacattcctacttctccttaacatttggcgtacaggtccagggcaagaacaacagagaaaagaaagacagactctccgtggacaagacctcagccaaggtcgtgtgccacaaggatgccaagatccgcgtgcaagccagagaccgctactatagctcatcctggagcaactgggcatccgtgtcctgcagttag

SEQ ID NO: 11 - DNA, 1929 bp, Feline leukemia virus
  misc_feature (1)..(1929): FeLV env-gp85 gene
atggaaagtccaacgcacccaaaaccctctaaagataagactctctcgtggaacttagtgtttctggtggggatcttattcacaatagacataggaatggccaatcctagtccacaccaaatatataatgtaacttgggtaataaccaatgtacaaactaacacccaagctaatgccacctctatgttaggaaccttaaccgatgtctaccctaccctacatgttgacttatgtgacctagtgggagacacctgggaacctatagtcctaagcccaaccaatgtaaaacacggggcacgttacccttcctcaaaatatggatgtaaaactacagatagaaaaaaacagcaacagacataccccttttacgtctgccccggacatgccccctcgctggggccaaagggaacacattgtggaggggcacaagatgggttttgtgccgcatggggatgtgaaaccaccggagaagcttggtggaagccctcctcctcatgggactatatcacagtaaaaagagggagtagtcaggacaataactgtgagggaaaatgcaaccccctgattttgcagttcacccagaaggggaaacaagcctcttgggacggacctaagatgtggggattgcgactataccgtacaggatatgaccctatcgccttattcacggtatcccggcaggtgtcaaccattacgccgcctcaggcaatgggaccaaacctagtcttacctgatcaaaaacccccatcccgacaatctcaaacagggtccaaagtggcgacccagaggccccaaacgaatgaaagcgccccaaggtctgttgcccccaccaccgtgggtcccaaacggattgggaccggagataggttaataaatttagtacaagggacatacctagccttaaatgccaccgaccccaacaaaactaaagactgttggctctgcctggtttctcgaccaccctattacgaagggattgcaatcttaggtaactacagcaaccaaacaaaccctcccccatcctgcctatctattccgcaacacaagctgaccatatctgaagtatcagggcaaggactgtgcatagggactgttcctaagacccaccaggctttgtgcaataagacgcaacagggacatacaggggcgcactatctagccgcccccaatggcacctattgggcctgtaacactggactcaccccatgcatttccatggcggtgctcaattggacctctgatttttgtgtcttaatcgaattatggcccagagtgacttaccatcaacccgaatatgtgtacacacattttgccaaagctgtcaggttccgaagagaaccaatatcactaactgttgccctcatgttgggaggactcactgtagggggcatagccgcgggggtcggaacagggactaaagccctccttgaaacagcccagttcagacaactacaaatggccatgcacacagacatccaggccctagaagagtcaattagtgccttagaaaagtccctgacctccctttctgaagtagtcttacaaaacagacggggcctagatattctattcctacaagagggagggctctgtgccgcattaaaagaagaatgttgcttctatgcggatcacaccggactcgtccgagacaatatggctaaattaagagaaagactaaaacagcggcaacaactgtttgactcccaacagggatggtttgaaggatggttcaacaggtccccctggtttacaaccctaatttcctccattatgggccccttactaatcctactcctaattctcctcttcggcccatgcatccttaacagattagtacaattcgtaaaagacagaatatctgtggtacaagccttaattttaacccaacagtaccaacagataaagcaatacgatccggaccgaccatga

SEQ ID NO: 12 - DNA, 1881 bp, Feline leukemia virus
  misc_feature (1)..(1881): FeLV gag-pr65-pro gene
atgggccaaactataactacccccttaagcctcacccttgatcactggtctgaagtccgggcacgagcccataatcaaggtgtcgaggtccggaaaaagaaatggattaccttatgtgaggccgaatgggtgatgatgaatgtgggctggccccgagaaggaactttttctcttgataacatttcccaggttgagaaaaagatcttcgccccgggaccgtatggacaccccgaccaagttccgtacattaccacatggagatccttagccacagaccccccttcgtgggttcgtccgttcctaccccctcccaaaactcccacacccctccctcaacctctatcgccgcagccctccgcccctcttacctcttccctctaccccgttctccccaagtcagaccctcccaaaccgcctgtgttaccgcctgatccttcttcccctttaattgatctcttaacagaagagccacctccctatccggggggtcacgggccaccgccatcaggtcctagaaccccaaccgcttccccgattgccagccggctaagggaacgacgagaaaaccctgctgaagaatctcaagccctccccttgagggaaggccccaacaaccggccccagtattggccattctcagcttcagacctgtataactggaagtcgcataacccccctttctcccaagaccccgtggccctaactaacctaattgagtccattttagtgacgcatcaaccaacctgggacgactgccagcagctcttgcaggcactcctgacaggcgaagaaaggcaaagggtccttcttgaggcccgaaagcaggttccaggcgaggacggacggccaacccagctgcccaatgtcattgacgaagctttccccttgacccgtcccaactgggattttcgtacgccggcaggtagggagcacctacgcctttatcgccagttgctgttagcgggtctccgcggggctgcaagacgccccactaatttggcacaggtaaagcaagttgtacaagggaaagaggaaacgccagcctcattcttagaaagattaaaagaggcttacagaatgtatactccctatgaccctgaggacccagggcaggctgctagtgttatcctgtcctttatctaccagtctagcccggacataagaaataagttacaaaggctagaaggcctacaggggttcacactgtctgatttgctaaaagaggcagaaaagatatacaacaaaagggagaccccagaggaaagggaagaaagattatggcagcggcaggaagaaagagataaaaagcgccataaggagatgactaaagttctggccacagtagttgctcagaatagagataaggatagagaggaaagtaaactgggagatcaaagaaaaatacctctggggaaagaccagtgtgcctattgcaaggaaaagggacattgggttcgcgattgccccaaacggccccggaagaaacccgccaactccactctcctcaacttagaagattaggagagtcagggccaggaccccccccctgagcccaggataaccttaaaaataggggggcaaccggtgactttcctggtggacacgggagcccagcactcagtattaactcgaccagatggacctctcagtgaccgcacagccctggtgcaaggagccacgggaagcaaaaactaccggtggaccaccgacaggagggtacaactggcaaccggtaaggtgactcattcttttttatatgtacctgaatgtccctacccgttattaggaagagacctattaactaaacttaaggcccaaatccattttaccggagaaggggctaatgttgttgggcccaggggtttacccctacaagtcctt

SEQ ID NO: 13 - DNA, 744 bp, Feline leukemia virus
  misc_feature (1)..(744): FeLV p27 gene
cccttgagggaaggccccaacaaccggccccagtattggccattctcagcttcagacctgtataactggaagtcgcataacccccctttctcccaagaccccgtggccctaactaacctaattgagtccattttagtgacgcatcaaccaacctgggacgactgccagcagctcttgcaggcactcctgacaggcgaagaaaggcaaagggtccttcttgaggcccgaaagcaggttccaggcgaggacggacggccaacccagctgcccaatgtcattgacgaagctttccccttgacccgtcccaactgggattttgctacgccggcaggtagggagcacctacgcctttatcgccagttgctgttagcgggtctccgcggggctgcaagacgccccactaatttggcacaggtaaagcaagttgtacaagggaaagaggaaacgccagcctcattcttagaaagattaaaagaggcttacagaatgtatactccctatgaccctgaggacccagggcaggctgctagtgttatcctgtcctttatctaccagtctagcccggacataagaaataagttacaaaggctagaaggcctacaggggttcacactgtctgatttgctaaaagaggcagaaaagatatacaacaaaagggagaccccagaggaaagggaagaaagattatggcagcggcaggaagaaagagataaaaagcgccataaggagatgactaaagttctg

SEQ ID NO: 14 - DNA, 2847 bp, Feline viral rhinotracheitis virus
  misc_feature (1)..(2847): glycoprotein B
atgtccactcgtggcgatcttgggaagcggcgacgagggagtcgttggcagggacacagtggctattttcgacagagatgttttttcccttctctactcggtattgcagcgactggctccagacatggtaacggatcgtcgggattaaccagactagctagatatgtttcatttatctggatcgtactattcttagtcggtccccgtccagtagagggtcaatctggaagcacatcggaacaaccccggcggactgtagctacccctgaggtagggggtacaccaccaaaaccaactacagatcccaccgatatgtcggatatgagggaagctctccgtgcgtcccaaatagaggctaacggaccatcgactttttatatgtgtccaccaccttcaggatctactgtcgtgcgtttagagccaccacgggcctgtccagattataaactagggaaaaattttaccgagggtatagctgtaatatttaaagaaaatatagcgccatataaattcaaggcaaatatatactataaaaacattattatgacaacggtatggtctgggagttcctatgccgttacaaccaaccgatatacagacagggttcccgtgaaagttcaagagattacagatctcatagatagacggggtatgtgcctctcgaaagctgattacgttcgtaacaattatcaatttacggcctttgatcgagacgaggatcccagagaactgcctctgaaaccctccaagttcaacactccagagtcccgtggatggcacaccaccaatgaaacatacacaaagatcggtgctgctggatttcaccactctgggacctctgtaaattgcatcgtagaggaagtggatgcaagatctgtatatccatatgactcatttgctatctccactggtgacgtgattcacatgtctccattctttgggctgagggatggagcccatgtagaacatactagttattcttcagacagatttcaacaaatcgagggatactatccaatagacttggatacgcgattacaactgggggcaccagtttctcgcaattttttggaaactccgcatgtgacagtggcctggaactggaccccaaagtgtggtcgggtatgtaccttagccaaatggagggaaatagatgaaatgctacgcgatgaatatcagggctcctatagatttacagtcaagaccatatccgctactttcatctccaatacttcacaatttgaaatcaatcgtatccgtttgggggactgtgccaccaaggaggcagccgaagccatagaccggatttataagagtaaatatagtaaaactcatattcagactggaaccctggagacctacctagcccgtggcggatttctaatagctttccgtcccatgatcagcaacgaactagcaaagttatatatcaatgaattagcacgttccaatcgcacggtagatctcagtgcactcctcaatccatctggggaaacagtacaacgaactagaagatcggtcccatctaatcaacatcataggtcgcggcgcagcacaatagaggggggtatagaaaccgtgaacaatgcatcactcctcaagaccacctcatctgtggaattcgcaatgctacaatttgcctatgactacatacaagcccatgtaaatgaaatgttgagtcggatagccactgcctggtgtacacttcagaaccgcgaacatgtgctgtggacagagaccctaaaactcaatcccggtggggtggtctcgatggccctagaacgtcgtgtatccgcgcgcctacttggagatgccgtcgccgtaacacaatgtgttaacatttctagcggacatgtctatatccaaaattctatgcgggtgacgggttcatcaacgacatgttacagccgccctcttgtttccttccgtgccctcaatgactccgaatacatagaaggacaactaggggaaaacaatgaccttctcgtggaacgaaaactaattgagccttgcactgtcaataataagcggtattttaagtttggggcagattatgtatattttgaggattatgcgtatgtccgtaaagtcccgctatcggagatagaactgataagtgcgtatgtggatttaaatcttactctcctagaggatcgtgaatttctcccactcgaagtttatacacgagctgagctggaagataccggccttttggactacagcgagattcaacggcgcaaccaactccacgccttaaaattttatgatatagacagcatagtcagagtggataataatcttgtcatcatgcgtggtatggcaaatttttttcagggactcggggatgtgggggctggtttcggcaaggtggtcttaggggctgcgagtgcggtaatctcaacagtatcaggcgtatcatcatttctaaacaacccatttggagcattggccgtgggactgttaatattagctggcatcgtcgcagcattcctggcatatcgctatatatctagattacgtgcaaatccaatgaaagccttatatcctgtgacgactaggaatttgaaacagacggctaagagccccgcctcaacggctggtggggatagcgacccgggagtcgatgacttcgatgaggaaaagctaatgcaggcaagggagatgataaaatatatgtccctcgtatcggctatggagcaacaagaacataaggcgatgaaaaagaataagggcccagcgatcctaacgagtcatctcactaacatggccctccgtcgccgtggacctaaataccaacgcctcaataatcttgatagcggtgatgatactgaaacaaatcttgtctaa

SEQ ID NO: 15 - DNA, 1125 bp, Feline viral rhinotracheitis virus
  misc_feature (1)..(1125): glycoprotein D
atgatgacacgtctacatttttggtggtgtggaatctttgcggtcctgaaatatctggtatgtacttcaagccttacgaccacgccaaaaacaactacggtttatgtgaagggatttaatatacctccactacgctacaattatactcaagccagaatcgtgccaaaaattccccaggcgatggacccgaagataacagctgaagtacgttatgtaacatcaatggattcatgtgggatggtggcattgatatcagagccggatatagacgctactattcgaaccatacaactatctcaaaaaaaaacatataacgcgactataagttggtttaaggtaacccagggttgtgaataccctatgtttcttatggatatgagactttgtgatcctaaacgggaatttggaatatgtgctttacggtcgccttcatattggttggaacctttaacaaagtatatgttcctaacagacgatgaactgggtttgattatgatggccccggcccaatttaatcaaggacaatatcgaagagttataaccatcgatggttccatgttttatacagattttatggtacaactatctccaacgccatgttggttcgcaaaacccgatagatacgaagagattctacatgaatggtgtcgaaatgttaaaactattggccttgatggagctcgtgattaccactattattgggttccctataacccacaacctcaccataaagccgtactcttatattggtatcggactcatggccgagaacccccagtaagattccaagaggccattcgatatgatcgtcccgccataccgtctgggagtgaggattcgaaacggtccaacgactcaagaggagaatcgagtggacccaattggatagacattgaaaattacactcctaaaaataatgtgcctattataatatctgacgatgacgttcctacagcccctcccaagggcatgaataatcagtcagtagtgatacccgcaatcgtactaagttgtcttataatagcactgattctaggagtgatatattatattttgagggtaaagaggtctcgatcaactgcatatcaacaacttcctataatacatacaactcaccatccttaa

(19) United States
(12) Patent Application Publication
(10) Pub. No.: US 2008/0299149 A1
(43) Pub. Date: Dec. 4, 2008

(54) Raccoon Poxvirus Expressing Genes of Feline Antigens

(75) Inventors: Stephen Qitu Wu, Fort Dodge, IA (US); Michael A. Gill, Fort Dodge, IA (US); Hsien-Jue Chu, Bonner Springs, KS (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/128,329

(22) Filed: May 28, 2008

Related U.S. Application Data
(60) Provisional application No. 60/932,419, filed on May 30, 2007.

(51) Int. Cl. A61K 39/00; C12N 15/00; A61P 37/00

(52) U.S. Cl. 424/199.1; 435/320.1

(57) ABSTRACT

The present invention relates to new recombinant raccoon poxvirus vectors comprising two or more exogenous nucleic acid molecules, each encoding at least one feline protein, wherein at least two of the nucleic acid molecules are inserted into the hemagglutinin (ha) locus or the thymidine kinase (tk) locus, or at least one of the nucleic acid molecules is inserted into each of the hemagglutinin and thymidine kinase loci. Described herein are monovalent and polyvalent recombinant feline vaccines that encompass an immunologically effective amount of the recombinant raccoon poxvirus vectors and, optionally, a suitable carrier or diluent. The vaccine of this invention optionally includes additional feline antigens to provide broad spectrum protection to cats against a variety of feline pathogens. The invention further concerns the method for inducing a protective immune response to the feline pathogens in a cat by administering the recombinant vaccines.

### CROSS-REFERENCE TO RELATED APPLICATIONS

This application claims the benefit under 35 U.S.C. §119 (e) of U.S. Provisional Application Ser. No. 60/932,419, filed May 30, 2007, the disclosure of which is incorporated by reference herein in its entirety.

### FIELD OF THE INVENTION

The present invention concerns new recombinant raccoon poxvirus vectors that express multiple genes of feline antigens and the use of the vectors in multivalent vaccines in the prophylaxis of infections or diseases caused by the feline pathogens.

### BACKGROUND OF THE INVENTION

Many feline infectious diseases become endemic and create catastrophic situations in multiple-cat environments, particularly animal hospitals, breeding catteries and, to a lesser extent, animal shelters. Two pathogens of great significance to the health of cats have been the feline calicivirus (FCV) and feline viral rhinotracheitis virus (FVR) since FVR and FCV comprise almost 90% of all feline respiratory infections. Typically, the FCV infection presents signs resembling viral rhinotracheitis (FVR) by affecting the upper respiratory tract and, on occasion, producing joint pain and lameness. Additionally, the infected cat will develop ulcers on the tongue and in the mouth region. Vesicles and erosions of the nasal passages, the hard palate and the tongue appear prevalent. Other symptoms of FCV disease include high fever, hair loss, skin ulcerations and edema (swelling) in the legs or around the face. Depending on the virulence of the infecting strain, the FCV infection may become fatal. The primary method of transmission is through the oral route of infection but cats can get the infection from inhalation of infectious virus found in the saliva, feces or urine of infected cats. FCV is highly contagious; infected cats will continue to shed the virus for long periods of time after infection and recovered cats may remain lifelong carriers of the infectious virus. Asymptomatic cats can even spread fatal disease to other healthy cats. Recent outbreaks have been reported in Northern Calif. and New England of two genetically diverse strains of highly virulent, hemorrhagic calicivirus that were particularly fatal to the feline population in animal shelters, named FCV-Ari and FCV-Diva, respectively (N.C. Pedersen et al., “An isolated epizootic of hemorrhagic-like fever in cats caused by a novel and highly virulent strain of feline calicivirus,” Veterinary Microbiol. 73:281-300 (May 2000); E. M. Schorr-Evans et al., An epizootic of highly virulent feline calicivirus disease in a hospital setting in New England,” Journal of Feline Medicine and Surgery 5.217-226 (2003)).

The feline viral rhinotracheitis virus (FVR) is a feline herpesvirus 1 (FHV-1), of the family Herpesviridae. FVR, found worldwide among domestic and wild cats, causes an infectious, acute, upper respiratory infection of cats, characterized by rhinitis (inflammation of the nose), fever, conjunctivitis (inflammation of the membrane lining the eyelid), nasal and ocular discharges and sneezing. The virus also affects the reproductive tract and can trigger complications during pregnancy, The viral illness is often known as rhinotracheitis (or feline herpesvirus infection) but also commonly known as feline influenza or coryza. The FVR respiratory disease in cats is highly contagious and can be serious, particularly in catteries. While all members of the Felidae family are susceptible to FVR, young kittens and old cats are more susceptible to severe disease caused by FVR, including death from pneumonia. It has been found that certain breeds such as Siamese and Burmese are more severely affected by FVR than others.

In addition to FCV, rhinotracheitis caused by FVR is part of the feline upper respiratory infection or disease complex, which is a group of viral and bacterial infections that cause sneezing along with the nasal and ocular discharges. Cats frequently catch two or more of the upper respiratory infections at the same time. Although FCV and FVR (FHV-1) cause the two most common infections, the respiratory disease complex also regularly includes chlamydiosis.

Feline chlamydiosis is caused by a worldwide spread pathogen Chlamydophila felis (formerly known as feline Chlamydia psittaci). Sometimes referred to as Chlamydia psittaci feline pneumonitis agent, the bacterial pathogen is the causative agent of conjunctivitis as well as pneumonia (pneumonitis) in cats. Even though conjunctivitis is often the major clinical symptom, the ailing cats may also experience mild sneezing and nasal discharge. At times, there is a mild fever resulting in lethargy and loss of appetite but usually, the cats infected with Chlamydophila felis appear well initially. If left untreated, however, the conjunctivitis generally persists for eight or more weeks and cats will shed the organism for several months. The infection can then progress to the more severe case of pneumonia.

Another serious pathogen causing contagious and deadly infections in cats is the feline leukemia virus (FeLV). Infection with FeLV is a common and major cause of fatal illness in domestic cats, being responsible for more deaths among cats than any other infectious disease. Cats may not begin to show signs of disease for months or even years after becoming infected with the virus, Once they become persistently (permanently) infected with FeLV, the cats are at high risk of developing serious illnesses of anemia and cancer. A female retrovirus made up of RNA and related to the feline immunodeficiency virus (FIV), FeLV is the causative agent of feline leukemia (a cancerous disease), immunodeficiency and other cancers. Between approximately 80-90% of affected cats die within three and a half years after being diagnosed with FeLV infection. Typically, the FeLV infection results in immunosuppression in which the virus attacks the cells of the immune system, By killing or damaging the white blood cells, the virus leaves the cat susceptible to a large variety of other diseases and secondary infections. FeLV infection is not highly contagious but rather, the spread of the virus relies on close and prolonged contact of cats, for example, catteries, animal shelters, multi-cat households and densely populated city cats where viral infection can infect up to 30% of the cats.

Hence, FVR and FCV comprise the vast majority of all feline respiratory ailments; FVR, FCV and feline chlamydiosis frequently infect the same cat as a group known as the feline upper respiratory disease complex; and infection with FeLV is often fatal. The development of an effective, combination vaccine to prevent these serious infections or deadly disease states in cats would be of great significance to the veterinary art.

In the past, monovalent vaccines have been described and several manufactured to prevent feline diseases using a variety of antigens such as the feline calicivirus F9 strain (U.S. Pat. No. 3,944,469), feline Chlamydia psittaci (U.S. Pat. Nos. 5,972,350 and 5,242,686), feline leukemia virus (U.S. Pat. No. 4,264,587) and the like. Other calicivirus strains such as the FCV-M8 and FCV-255 and feline rhinotracheitis virus have also been previously isolated and described for vaccine use (E. V. Davis et al., “Studies on the safety and efficacy of an intranasal feline rhinotracheitis-calici virus vaccine,” VM-SAC 71:1405-1410 (1976); D. E. Kahn et al., “Induction of immunity to feline caliciviral disease,” Infect. Immun. 11:1003-1009 (1975); D. E. Kahn, “Feline viruses: pathogenesis of picornavirus infection in the cat,” Am. J. Vet. Research 32:521-531 (1971)). U.S. Pat. No. 6,231,863 describes nucleotide sequences from the genome of the FCV-2280 strain and vaccines using the nucleotide sequences of the capsid gene for preventing feline calicivirus disease. U.S. Pat. No. 5,106,619 discloses the preparation of inactivated viral vaccines that include feline calicivirus among others. U.S. Pat. No. 6,051,239 describes oral vaccines that use a modified botulinum toxin in conjunction with antigens such as the calicivirus.

Certainly, multivalent vaccines provide advantages over the older monovalent vaccines in being able to inoculate the cat against a wide group of pathogens, which would be less traumatic to the cats and easier for the cat handler or veterinarian. Multivalent vaccines have thus been prepared or described to contain mixtures of many antigens such as Chlamydophila felis (formerly known as feline Chlamydia psittaci) in combination with one or more pathogens comprising feline leukemia virus, feline panleukopenia virus, feline calicivirus, feline rhinotracheitis virus, feline acquired immunodeficiency virus, rabies, feline infectious peritonitis, Borrelia burgdorferi and the like (U.S. Pat. No. 6,004,563). Another mixture of Rickard isolate feline leukemia virus, feline rhinotracheitis virus, feline calicivirus and feline panleukemia virus has similarly been disclosed as a vaccine (U.S. Pat. No. 5,374,424).

Unfortunately, none of the prior vaccines that contain previously used strains of the feline calicivirus adequately protect the feline from the emerging hemorrhagic feline calicivirus strains. In the recent hemorrhagic feline calicivirus outbreaks, there were a significant number of deaths despite the fact that the cats had received vaccinations against the calicivirus. Moreover, the vaccination of cats presents its own unique difficulties in that cats sometimes have idiosyncratic reactions to certain pathogens and sarcoma-induced side effects to typical injectable formulations that require the addition of adjuvants to obtain sufficient immune response to the inoculant.

Attempting to improve feline vaccine compositions for better cat immunity against serious feline infections or diseases, research efforts have been directed toward recombinant technology. To date, there is a significant amount of published information on the topic of recombinant raccoon poxvirus as vaccines. Nevertheless, it is a complex task to find and develop a functional, multivalent recombinant vaccine that successfully and adequately expresses antigenic proteins for sufficient immune response in the cat while avoiding the necessity to include adjuvants.

For instance, U.S. Pat. No. 6,241,989 and its continuation U.S. Pat. No. 7,087,234 deal with multivalent recombinant raccoon poxviruses, containing more than one exogenous gene inserted into either the thymidine kinase gene or the hemagglutinin gene. Disclosed in these two related patents is the use of the multivalent recombinant raccoon poxviruses as vaccines to immunize felines against subsequent challenge by feline pathogens. Also disclosed is a method of making a multivalent recombinant raccoon poxvirus by a recombinant process involving the construction of an insertion vector into which the exogenous genes are inserted; and flanking the inserted genes are sequences which can recombine into the raccoon poxvirus thymidine kinase gene or the hemagglutinin gene; introducing both the insertion vector containing the exogenous genes, and raccoon poxvirus into susceptible host cells; and selecting the recombinant raccoon poxvirus from the resultant plaques. The multivalent, recombinant raccoon poxvirus of the patents can infect and replicate in feline cells, and contains more than one exogenous gene inserted into a region consisting of a hemagglutinin gene or a thymidine kinase gene of the raccoon poxvirus genome which is non-essential for viral replication, notably wherein the exogenous genes are operably linked to a promoter for expression; and each exogenous gene encodes a feline pathogen antigen. The patents describe exogenous genes encoding feline pathogen antigens such as feline leukemia virus (FeLV Env), feline immunodeficiency virus (FIV Gag), feline immunodeficiency virus (FIV Env), feline infectious peritonitis virus (FIPV M), feline infectious peritonitis virus (FIPV N), feline calicivirus (FCV capsid protein), feline panleukopenia virus (FPV VP2) and rabies-G.

U.S. Pat. No. 6,294,176 concerns a recombinant raccoonpox virus (RCNV) vaccine that consists of a raccoonpox virus viral genome which contains a foreign DNA sequence inserted into a non-essential region within the HindIII “U” genomic region, the HindIII “M” genomic region or the HindIII “N” genomic region of the raccoonpox virus genome. The raccoonpox virus viral genome is described in the patent as containing a deletion in the raccoonpox virus host range gene of the viral genome. The patent provides a homology vector for producing the recombinant raccoonpox virus by inserting the foreign DNA sequence into the raccoonpox virus genome.

U.S. Pat. No. 6,106,841 relates specifically to a distinctive delivery method for immunizing an animal against a heterologous antigen. The method describes administering to the animal via the conjunctival route, a composition comprising a recombinant raccoon poxvirus having a nucleic acid molecule encoding the heterologous antigen. In addition to the conjunctival route, the patent also discloses the intranasal vaccination route of administration. Heterologous antigens that may be expressed by the recombinant raccoon poxvirus and used in the patented method are listed ascalicivirus, coronavirus, herpesvirus, immunodeficiency virus, infectious peritonitis virus, leukemia virus, parvovirus antigen, rabies virus, Bartonella, Yersinia, Dirofilaria, Toxoplasma, flea antigen or flea allergen, midge antigen or allergen, mite antigen or allergen and a tumor antigen. Additionally, the recombinant raccoon poxvirus may comprise a nucleic acid molecule encoding an immunomodulator such as cytokines, chemokines and other immunomodulators; however, there is no specific example of how such a construct would be generated. Furthermore, there is no disclosure of how to make any new recombinant raccoon poxviruses encoding heterologous antigens since patentees only use old constructs in the exemplification of their claimed method. There is one reference to known RCNV/PLA2 poxviruses where at least one of the nucleic acid molecules encodes a heartworm PLA2 antigen; and the working examples only demonstrate the intranasal and/or conjunctival administration of a known recombinant raccoon poxvirus expressing the rabies glycoprotein G (gG) protein, i.e., RCNV/rabies gG (RCN/G). Notably, the known RCNV/rabies gG construct is prepared by inserting within the thymidine kinase gene of the virus, a heterologous nucleic acid molecule encoding a rabies glycoprotein G protein operatively linked to a poxvirus p11 promoter. The patent does not describe or suggest making any other novel form of a recombinant raccoon poxvirus.

U.S. Pat. No. 6,010,703 concerns a recombinant poxvirus vaccine against feline herpesvirus (FHV-1) that provides immunity to FHV-1 in cats and can be used in a method for inhibiting feline viral rhinotracheitis (FVR) in felines. The patent describes a recombinant raccoon poxvirus containing and expressing a gene encoding the feline herpesvirus gD glycoprotein precursor polypeptide or a gene encoding a gB precursor peptide wherein the gene is inserted or cloned into the poxvirus-thymidine kinase donor plasmid. The raccoon pox recombinants, only expressing FHV-1 gB or FHV-1 gD, are illustrated as both inducing protection against clinical signs of the disease.

Additional recombinant technology has similarly been used for the expression of single feline antigens such as feline immunodeficiency virus (FIV) or feline infectious peritonitis virus (FIPV). For example, U.S. Pat. No. 5,989,562 relates to recombinant raccoon poxviruses useful in vaccines for the prophylaxis of disease caused by feline immunodeficiency virus (FIV). According to the patent's disclosure, the recombinant raccoon poxvirus has at least one internal gene comprising a DNA sequence that encodes the FIV gag protein (gag) of feline immunodeficiency virus (FIV), FIV envelope protein (env), a polypeptide consisting of amino acids 1-735 of FIV env, or immunogenic fragments thereof. The vaccines that comprise one or more of the FIV-expressing recombinant raccoon poxviruses described therein may also comprise a pharmaceutically acceptable carrier or diluent and a pharmaceutically acceptable adjuvant, U.S. Pat. No. 5,989,562 also provides methods for preventing or lessening disease caused by FIV, which is carried out by administering to a feline in need of such treatment the vaccines described above. Incorporation of the FIV gag or env gene into the poxvirus DNA is accompanied only by disruption of the viral thymidine kinase gene.

Similarly, U.S. Pat. No. 5,820,869 relates to a recombinant raccoon poxvirus that express the envelope protein of feline immunodeficiency virus (FIV) and is useful as a vaccine, either alone or in combination with carriers and adjuvants. More particularly, the patent describes a recombinant raccoon poxvirus having at least one internal gene comprising a DNA sequence encoding the envelope protein of FIV or immunogenic fragments therefrom.

U.S. Pat. No. 5,770,211 discloses a recombinant raccoon poxvirus that expresses the nucleocapsid and transmembrane proteins of feline infectious peritonitis virus (FIPV). A recombinant raccoon poxvirus having at least one internal gene comprising a DNA sequence encoding the transmembrane (M/E1) protein of FIPV is specifically described and claimed in the patent.

U.S. Pat. No. 5,656,275 also describes a recombinant raccoon poxvirus that expresses the nucleocapsid and transmembrane proteins of feline infectious peritonitis virus (FIPV). A recombinant raccoon poxvirus having at least one internal gene comprising a DNA sequence encoding the nucleocapsid (N) protein of FIPV is specifically described and claimed in the patent.

U.S. Pat. No. 5,505,941 concerns a method for inducing an immunological response in a mammal or avian host to a pathogen by inoculating the mammal or avian host with a synthetic recombinant avipox virus, such as fowlpox virus or canarypox virus, modified by the presence, in a non-essential region of the avipox genome, of DNA from any source which codes for and expresses an antigen of the pathogen. The patent identifies antigens selected from the group consisting of rabies G antigen, gp51,30 envelope antigen of bovine leukemia virus, FeLV envelope antigen of feline leukemia virus and glycoprotein D antigen of herpes simplex virus. Specifically, the patent shows the construction of an avipox virus recombinant that expresses the feline leukemia virus (FeLV) envelope (env) of glycoprotein in which the FeLV env gene contains the sequences which encode the p70+p15E polyprotein. This gene was inserted into the plasmid with the vaccinia H6 promoter juxtaposed 5′ to the FeLV env gene where the plasmid was derived by first inserting an 1802 bp Sal I/Hind III fragment containing the vaccinia hemagglutinin (ha) gene into a pUC18 vector.

Despite all the efforts made in the veterinary vaccine art, a definite art-recognized need still exists to provide a safe and efficacious combination vaccine that gives an adequate protective immune response in a cat against a wide range of feline antigens. Due to highly virulent, hemorrhagic feline calicivirus infections that are prevalent in animal shelters, multi-cat households and the like, another art-recognized need is to provide a broad-spectrum viral vaccine that protects cats against serious infection and disease caused by both hemorrhagic and common FCV strains. Yet another art-recognized need is to create a multivalent vaccine capable of eliciting a specific immune response against the virulent, hemorrhagic strain of FCV and other feline pathogens in order to protect cats from acute and chronic viral or bacterial disease. The feline multivalent vaccine of the present invention solves the technological problem existing in the art by uniquely achieving excellent antibody titers and making broad-spectrum immunization possible through a novel combination of at least six different fractions of recombinant constructs.

The foregoing objects are accomplished by providing a safe and effective recombinant feline comb (combination) vaccine as described herein in which the vaccine elicits a protective immune response in the cat to multiple feline antigens without the addition of adjuvants.

All patents and publications cited in this specification are hereby incorporated by reference in their entirety.

### SUMMARY OF THE INVENTION

In its broadest aspect, the present invention provides safe and effective, adjuvant-free, recombinant feline vaccines that are useful as monovalent or polyvalent vaccines using raccoon poxviruses as vectors for expressing multiple feline viral, bacterial and cytokine antigens at the hemagglutinin (ha) and/or the thymidine kinase (tk) insertion loci of the raccoon poxvirus genome. The novel raccoon poxvirus vectors are preferentially designed to possess at least one nucleic acid molecule inserted into the hemagglutinin locus or the thymidine kinase locus of the raccoon poxvirus genome; at least two nucleic acid molecules inserted into the hemagglutinin locus or the thymidine kinase locus of the raccoon poxvirus genome or, in the alternative, at least one nucleic acid molecule inserted into the hemagglutinin locus and, concomitantly, at least one nucleic acid molecule inserted into the thymidine kinase locus of the raccoon poxvirus genome. Specifically, the constructs express the nucleic acid molecule or gene encoding the feline calicivirus (FCV) capsid protein, feline viral rhinotracheitis virus (FVR) glycoproteins DIB (gD/gB), feline Chlamydia psittaci (FCP, now commonly known as Chlamydophila felis) outer membrane protein (momp), feline leukemia virus (FeLV) gag-pr65-pro/env-gp70/env-gp85, and feline interleukin-12 (IL-12) P35/P40, the latter component being included as an immunomodulator to enhance immunogenicity of the comb vaccine in cats. The monovalent and polyvalent recombinant feline vaccines of the present invention encompass an immunologically effective amount of the recombinant raccoon poxvirus vectors and, optionally, a suitable carrier or diluent. Beneficially, the comb vaccine formulation does not require adjuvants to enhance the host immune response thereby avoiding the adjuvant-related sarcoma side effect that can occasionally occur with some traditional injectable vaccines. The vaccine of this invention optionally includes the one or more additional feline antigens to provide broad spectrum protection to cats against a variety of feline pathogens. The invention further concerns the method for inducing a protective immune response to the feline pathogens in a cat by administering the recombinant vaccines.

Accordingly, a first aspect provides for a recombinant raccoon poxvirus vector (rRCNV) comprising two or more exogenous, homologous nucleic acid molecules, each encoding at least one feline protein from two or more different strains of the same feline pathogen, wherein at least two of the nucleic acid molecules are inserted into the hemagglutinin (ha) locus or the thymidine kinase (tk) locus, or at least one of the nucleic acid molecules is inserted into each of the hemagglutinin and thymidine kinase loci. When two exogenous nucleic acids are inserted into the same locus, they may be contiguous or they may be separated by intervening sequences.

In one embodiment, the recombinant raccoon poxvirus vector further comprises a nucleic acid molecule encoding a feline viral/bacterial antigen or protein that is inserted into any non-essential site of the raccoon poxvirus genome.

In one embodiment, the recombinant raccoon poxvirus vector further comprises a nucleic acid molecule encoding a feline viral/bacterial antigen or protein that is inserted into a third non-essential site of the raccoon poxvirus genome in addition to the thymidine kinase and the hemagglutinin loci of the raccoon poxvirus genome.

In one embodiment, the third non-essential site of the raccoon poxvirus genome is the serine protease inhibitor site.

In one embodiment, the raccoon poxvirus is live and replicable.

In one embodiment, the recombinant raccoon poxvirus vector comprises a nucleic acid molecule encoding a feline calicivirus capsid protein.

In one embodiment, the recombinant raccoon poxvirus vector comprises a nucleic acid molecule encoding the feline calicivirus capsid protein of FCV-2280, which is inserted into the hemagglutinin or the thymidine kinase locus of the raccoon poxvirus genome, but preferably the hemagglutinin locus.

In one embodiment, the recombinant raccoon poxvirus vector comprises the nucleotide sequence of the FCV-2280 capsid gene, which is operably linked to a vaccinia virus late promoter P₁₁ or an early-late promoter for expression, which promoter may be a synthetic early-late promoter for expression.

In one embodiment, the recombinant raccoon poxvirus vector further comprises a nucleic acid molecule encoding the feline calicivirus capsid protein of FCV-DD1 inserted into the hemagglutinin locus or the thymidine kinase of the raccoon poxvirus genome but preferably the hemagglutinin locus.

In one embodiment, the recombinant raccoon poxvirus vector further comprises the nucleotide sequence of the FCV-2280 capsid gene, which is operably linked to a vaccinia virus late promoter for expression and the nucleotide sequence of the FCV-DD1 capsid gene is operably linked to an early-late promoter for expression, which promoter may be a synthetic early-late promoter for expression.

In one embodiment, the recombinant raccoon poxvirus vector further comprises a nucleic acid molecule encoding the feline calicivirus capsid protein of FCV-255 inserted into the hemagglutinin locus or the thymidine kinase of the raccoon poxvirus genome but preferably the hemagglutinin locus.

In one embodiment, the recombinant raccoon poxvirus vector further comprises a nucleic acid molecule encoding the feline viral rhinotracheitis virus glycoprotein gD and a nucleic acid molecule encoding the feline viral rhinotracheitis virus glycoprotein gB, which are inserted into the hemagglutinin locus or the thymidine kinase of the raccoon poxvirus genome but preferably the hemagglutinin locus.

In one embodiment, the recombinant raccoon poxvirus vector further comprises a nucleic acid molecule encoding the feline leukemia virus antigen gag-pr65-pro, and a nucleic acid molecule encoding the feline leukemia virus antigen env-gp85, which are inserted into the hemagglutinin locus or the thymidine kinase of the raccoon poxvirus genome but preferably the hemagglutinin locus.

In one embodiment, the nucleotide sequences encoding the viral antigens in the raccoon poxvirus vector are operably linked to a synthetic early-late promoter for expression.

In one embodiment, the recombinant raccoon poxvirus vector further comprises a nucleic acid molecule encoding the feline leukemia virus antigen gag-pr65-pro, a nucleic acid molecule encoding the feline leukemia virus antigen env-gp70 and a nucleic acid molecule encoding the feline leukemia virus antigen env-gp85, which are inserted into the thymidine kinase locus or the thymidine kinase locus of the raccoon poxvirus genome or both loci. In one embodiment, the recombinant raccoon poxvirus vector further comprises a nucleic acid molecule encoding a Chlamydophila felis protein, which is inserted into the thymidine kinase locus or the hemagglutinin locus of the raccoon poxvirus genome but preferably the hemagglutinin locus.

In one embodiment, the recombinant raccoon poxvirus vector further comprises a nucleic acid molecule encoding the outer membrane protein of Chlamydophila felis, which is inserted into the thymidine kinase locus or the hemagglutinin locus of the raccoon poxvirus genome but preferably the hemagglutinin locus. In one embodiment, the nucleotide sequence of the outer membrane protein gene of Chlamydophila felis is operably linked to a vaccinia virus late promoter for expression.

In one embodiment, the recombinant raccoon poxvirus vector further comprises a nucleic acid molecule encoding the P35 protein of feline interleukin-12 and a nucleic acid molecule encoding the P40 protein of feline interleukin-12, which are inserted into the hemagglutinin locus of the raccoon poxvirus genome or the thymidine kinase locus of the raccoon poxvirus genome but preferably the hemagglutinin locus.

A second aspect of the invention provides a feline vaccine comprising an immunologically effective amount of the recombinant raccoon poxvirus vectors as described herein and, optionally, a suitable carrier or diluent.

In one embodiment, the raccoon poxvirus is live and replicable.

In one embodiment, the vaccine is administered as a single dose or as repeated doses.

In one embodiment, the vaccine is adjuvant-free.

In one embodiment, the invention provides a feline combination vaccine comprising an immunologically effective amount of two or more of the recombinant raccoon poxvirus vectored constructs expressing feline viral/bacterial antigens and/or cytokines such as IL-12 of the invention. The example of the combination vaccines includes but not limited: (1) rRCNV-Feline 3 (modified live FPV, rRCNV-FCV, rRCNV-FVR); (2) rRCNV-Feline 4 (rRCNV-Feline 3+rRCNV-FCP); (3) rRCNV-Feline 4+rRCNV-FeLV; (4) rRCNV-Feline IL-12 may be formulated in each combination vaccine as an immunomodulator.

In one embodiment, the vaccine further comprises a mixture of one or more additional feline antigens selected from the group consisting of feline panleukopenia virus, feline immunodeficiency virus, rabies virus, feline infectious peritonitis virus, Bartonella bacteria, FCV-Diva, FCV-Kaos, FCV-Bellingham, FCV-F9, FCV-F4, FCV-M8 and a combination thereof.

In one embodiment, the vaccine comprises a mixture of two or more of the recombinant raccoon poxvirus vectors selected from the group consisting of rRCNV-FCV2280, rRCNV-FCV2280-FCVDD1, rRCNV-FCV2280-FCVDD1-FCV255, rRCNV-FVR gD/gB, rRCNV-FeLV gag-pr65-pro/env-gp85, rRCNV-FeLV gag-pr65-pro/env-gp70/env-gp85, rRCNV-FCP momp and rRCNV-feline IL-12 P35/P40.

In one embodiment, the vaccine further comprises a mixture of one or more additional feline antigens selected from the group consisting of feline panleukopenia virus, feline immunodeficiency virus, rabies virus, feline infectious peritonitis virus, Bartonella bacteria, FCV-Diva, FCV-Kaos, FCV-Beilingham, FCV-F9, FCV-F4, FCV-M8 and a combination thereof.

A third aspect of the invention provides a method for inducing a protective immune response to a feline pathogen in a cat comprising administering to the cat an effective immunizing amount of at least one of the vaccines as described herein.

In one embodiment, the effective immunizing amount of the vaccine is at least about 4.5 Log₁₀TCID₅₀/ml.

In one embodiment, the effective immunizing amount of the vaccine ranges from about 4.5 Log₁₀TCID₅₀/ml to about 7.5 Log₁₀TCID₅₀/ml.

In one embodiment, the protective immune response is a humoral or antibody mediated response.

In one embodiment, the protective immune response is a cell-mediated or T cell mediated immune response.

A fourth aspect of the invention provides one or more of the nucleic acid sequences and plasmid constructs as described herein.

In one embodiment, a plasmid comprises any one of the nucleotide sequences of SEQ ID NOs: 1, 2, 3 or 4.

In some embodiments of the invention, nucleic acids encoding feline calicivirus capsid proteins are from specific strains such as FCV-2280 or FCV-DD1.

In other embodiments either of these strains may be replaced with a nucleic acid encoding the same or a similar protein from another strain of calicivirus. The other strain of calicivirus may or may not cross protect against either of FCV-2280, or FCV-DD1. For example, the nucleic acid encoding an FCV-DD1 can be replaced by another hypervirulent, virulent, hemorrhagic or virulent systemic strain, as well known in the art. For example, see U.S. Pat. No. 7,029,682; WO 2005/072214, US 2006/0057159, U.S. Pat. No. 6,541,458; U.S. Pat. No. 6,534,066 and U.S. 2004/0259225.

A fifth aspect of the invention provides a recombinant raccoon poxvirus vector (RCNV) comprising:
a) at least one exogenous nucleic acid and at least one homologous exogenous nucleic acid each encoding the same feline protein from two or more different strains of a feline pathogen, wherein the at least one homologous nucleic acid is inserted into at least one of either the ha locus, or the tk locus; or
b) at least two exogenous nucleic acids, each nucleic acid encoding at least one different feline protein from the same feline pathogen; wherein one of the exogenous nucleic acids is inserted into at least one of either the ha or the tk site, or, wherein at least one nucleic acid is inserted into the ha locus and at least one nucleic acid is inserted into the tk locus.

In one embodiment, the recombinant raccoon poxvirus vector (RCNV) comprises at least two homologous, exogenous nucleic acid molecules, each encoding the same feline protein from two or more different strains of a feline pathogen, wherein the homologous, exogenous nucleic acid molecules are inserted into the ha locus, the tk locus, the serine protease inhibitor locus, or wherein at least one homologous exogenous nucleic acid molecule is inserted into each of the ha, tk, or serine protease inhibitor loci.

In one embodiment, the recombinant raccoon poxvirus vector further comprises at least two exogenous nucleic acid molecules, wherein at least one of the at least two exogenous nucleic acid molecules encodes one different feline protein selected from the group consisting of a feline calicivirus protein, glycoprotein gB of feline rhinotracheitis, glycoprotein gD of feline rhinotracheitis, a gag protein from feline leukemia virus, an env protein from feline leukemia virus, a Chlamydophila felis protein, and a P35 and P40 protein of feline interleukin-12.

A sixth aspect of the invention provides for a vaccine or immunogenic composition comprising any one or more of the recombinant raccoon poxvirus vectors described above for administering to cats. The vaccine may further comprise a mixture of one or more additional feline antigens selected from the group consisting of feline panleukopenia virus, feline immunodeficiency virus, rabies virus, feline infectious peritonitis virus, Bartonella bacteria, FCV-Diva, FCV-Kaos, FCV-Bellingham, FCV-F9, FCV-F4, FCV-M8 and a combination thereof.

In one embodiment, the vaccines described above may be used to induce a protective immune response to a feline pathogen in a cat by administering to the cat an effective immunizing amount of the vaccines described.

In one embodiment, the effective immunizing amount of the vaccine is at least about 4.5 Log₁₀TCID₅₀/ml.

In one embodiment, the effective immunizing amount of the vaccine ranges from about 4.5 Log₁₀TCID₅₀/ml to about 7.5 Log₁₀TCID₅₀/ml.

### BRIEF DESCRIPTION OF THE DRAWINGS

FIG. 1 is the nucleic acid sequence of the pFD2000A-FDAH plasmid (SEQ ID NO: 1) FIG. 2 is the nucleic acid sequence of the pFD2001 TK-FDAH plasmid (SEQ ID NO: 2) FIG. 3 is the nucleic acid sequence of the pFD2003SEL-FDAH plasmid (SEQ ID NO: 3)

FIG. 4 is the nucleic acid sequence of the pFD2003SEL-GPV-PV-FDAH plasmid (SEQ ID NO: 4)

FIG. 5 is the nucleic acid sequence of FCP momp-FDAH (SEQ ID NO: 5) FIG. 6 is the nucleic acid sequence of FCV255-Bmut-N Deletion-FDAH (SEQ ID NO: 6)

FIG. 7 is the nucleic acid sequence of FCV2280-N-Deletion-FDAH (SEQ ID NO: 7) FIG. 8 is the nucleic acid sequence of FCVDD1-N Deletion-FDAH (SEQ ID NO: 8) FIG. 9 is the nucleic acid sequence of Feline IL-12 p35-FDAH (SEQ ID NO: 9) FIG. 10 is the nucleic acid sequence of Feline IL-12 P40-FDAH (SEQ ID NO: 10) FIG. 11 is the nucleic acid sequence of FeLV 61E Env-gp85-FDAH (SEQ ID NO: 11) FIG. 12 is the nucleic acid sequence of FeLV 61 E gag-pr65-pro-FDAH (SEQ ID NO: 12)

FIG. 13 is the nucleic acid sequence of FeLV 61 E P27-FDAH (SEQ ID NO: 13) FIG. 14 is the nucleic acid sequence of FVR-gB-FDAH (SEQ ID NO: 14) FIG. 15 is the nucleic acid sequence of FVR-gD-BKXMut-FDAH (SEQ ID NO: 15)

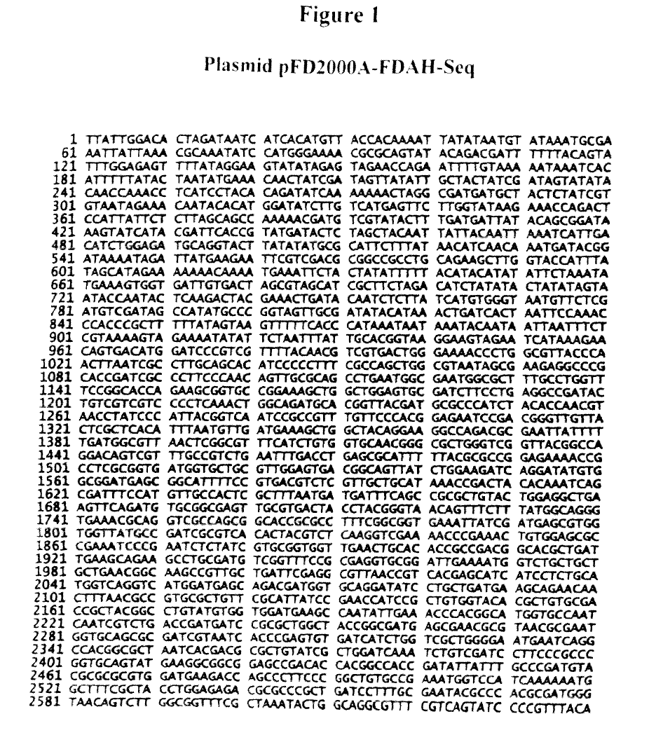

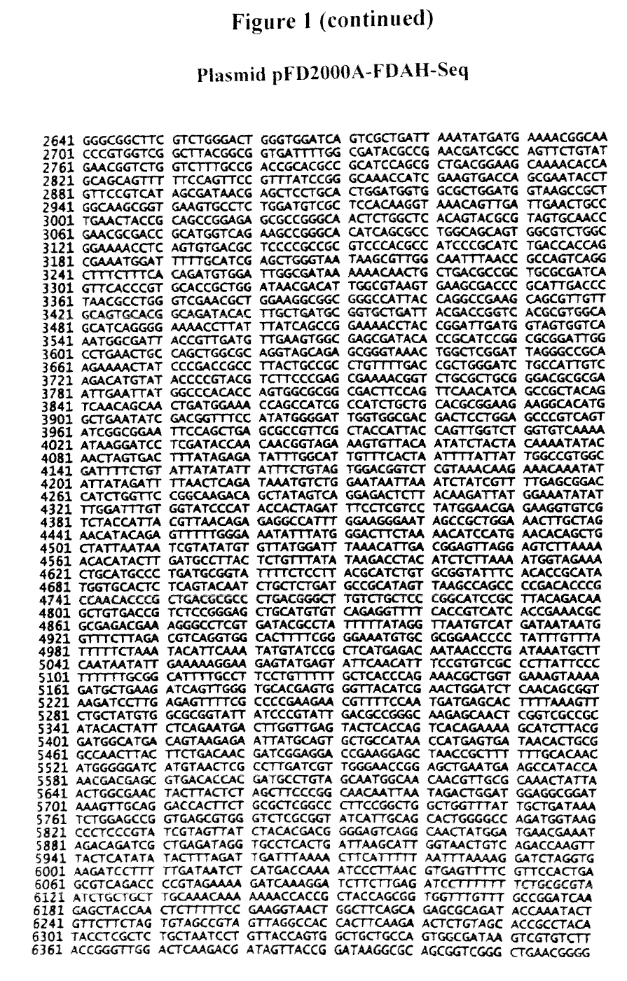

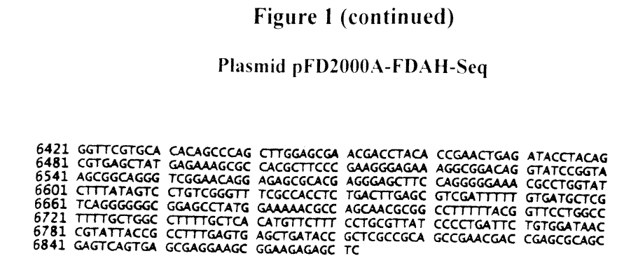

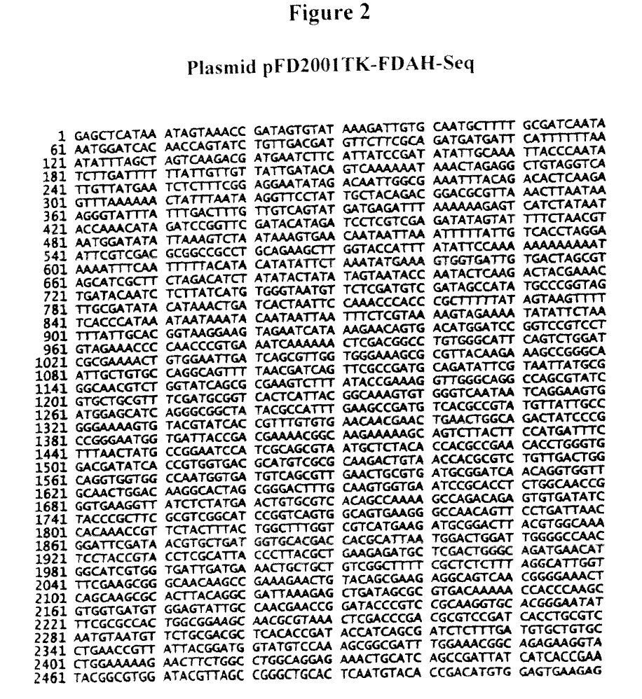

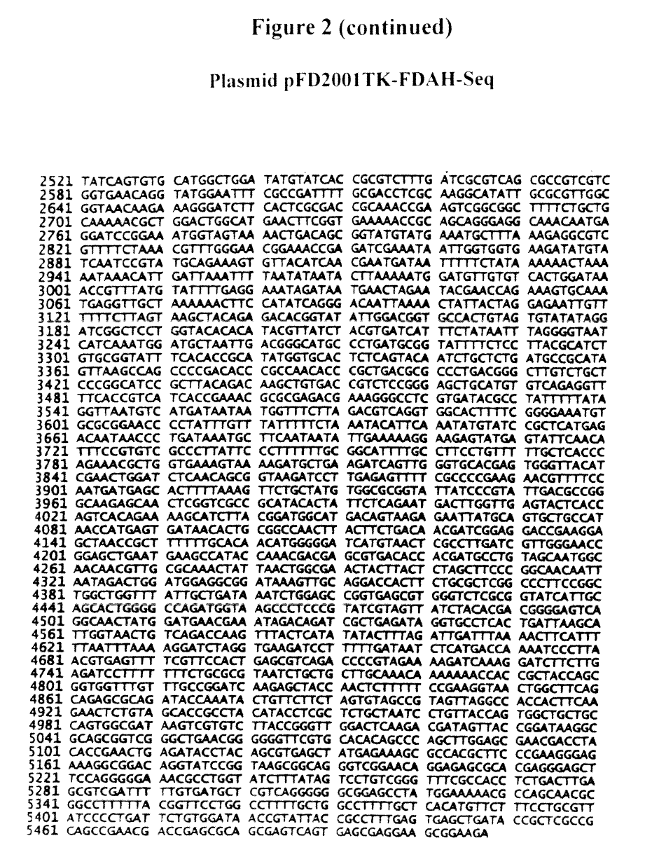

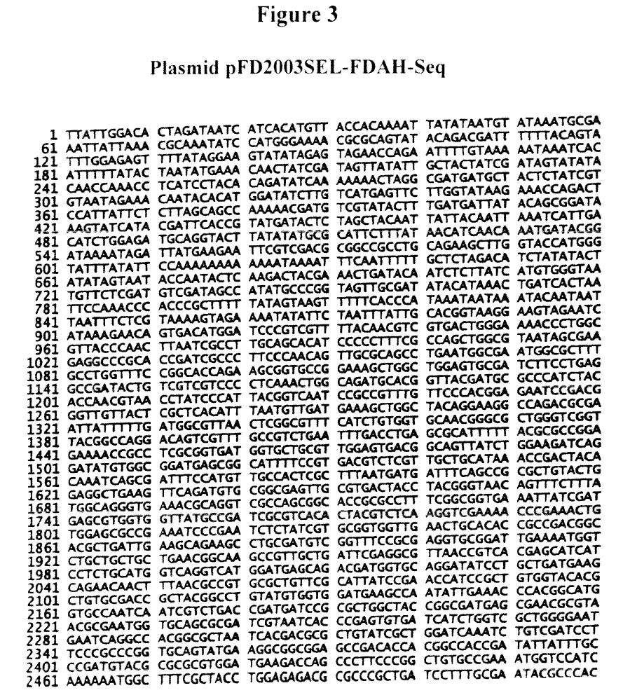

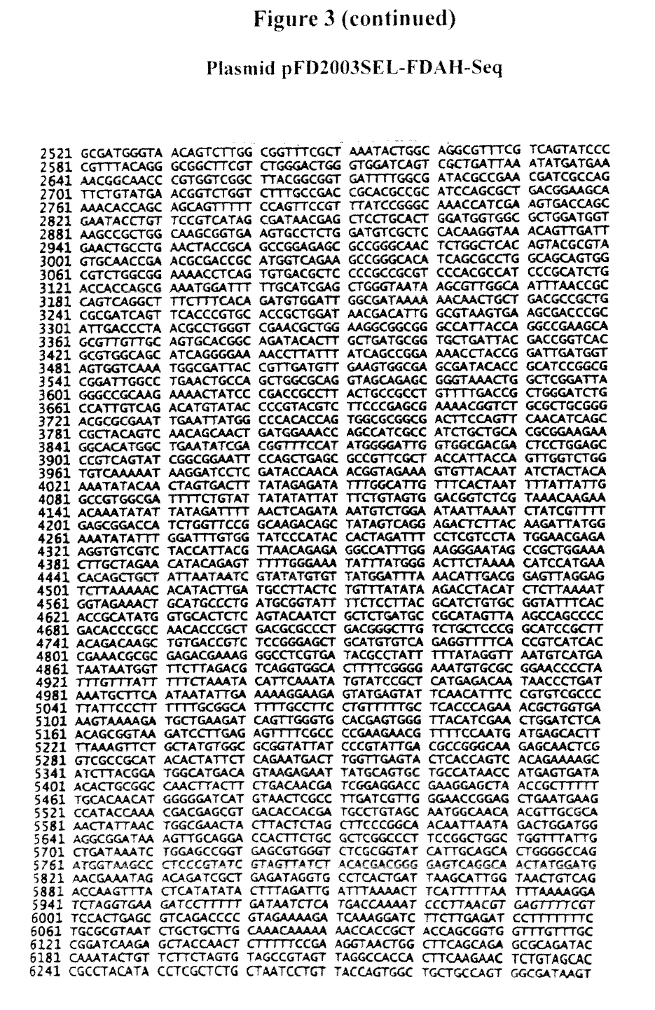

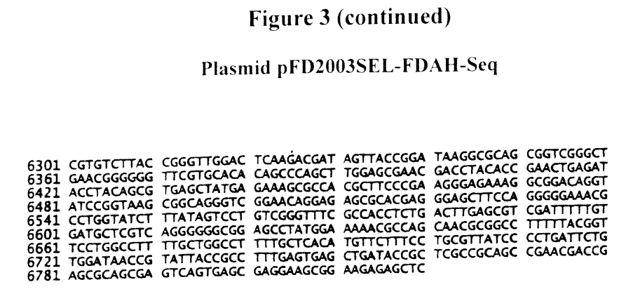

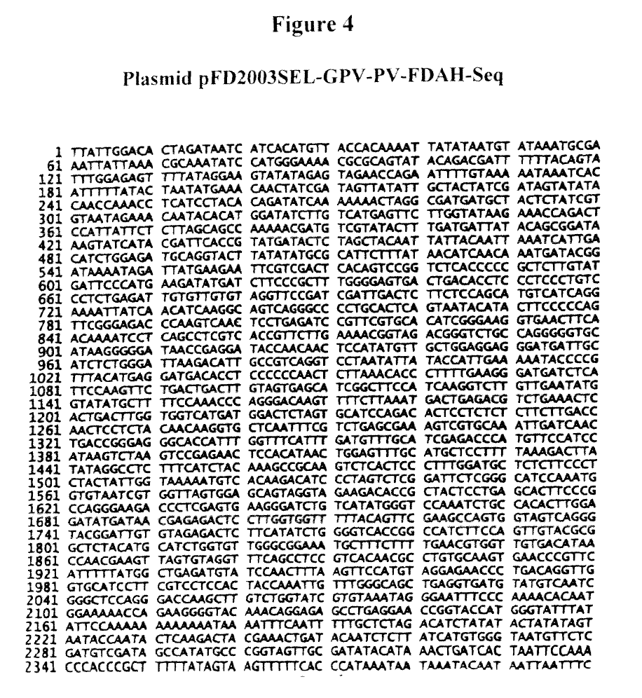

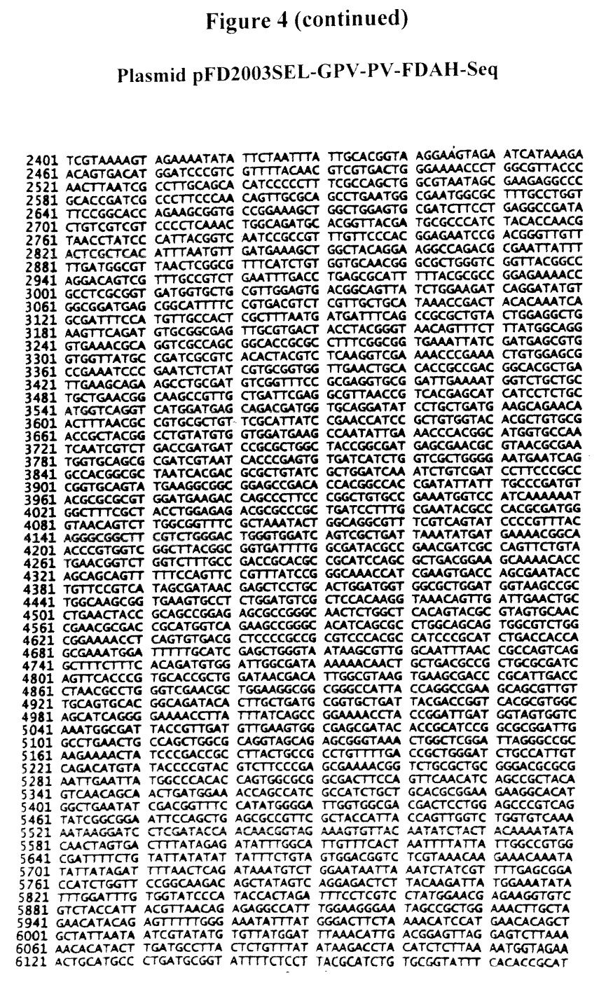

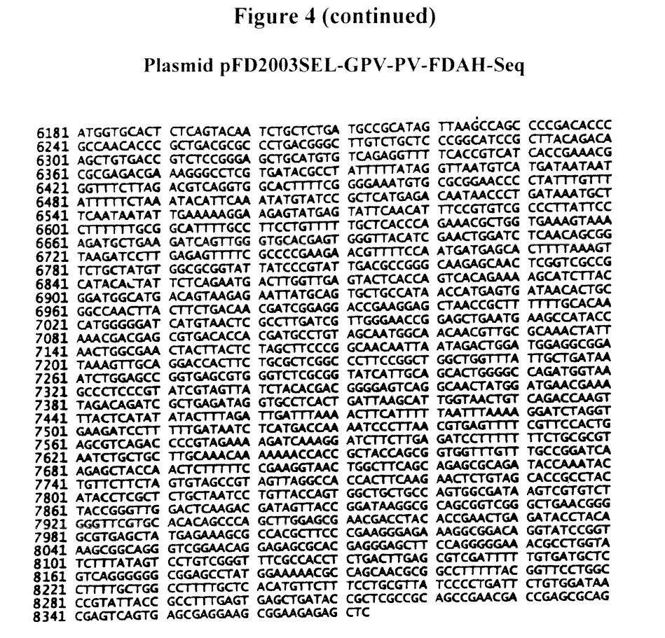

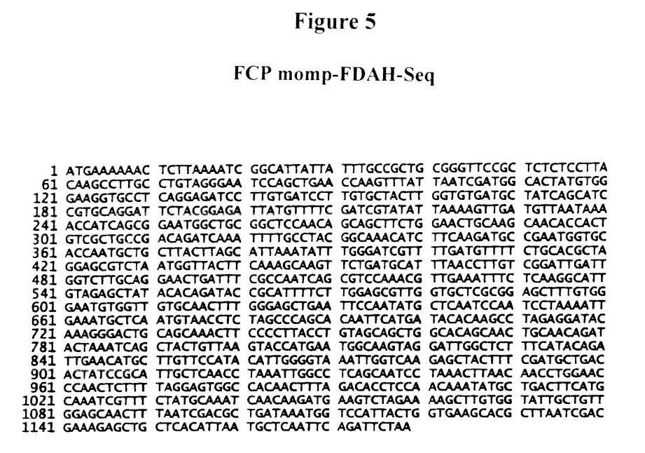

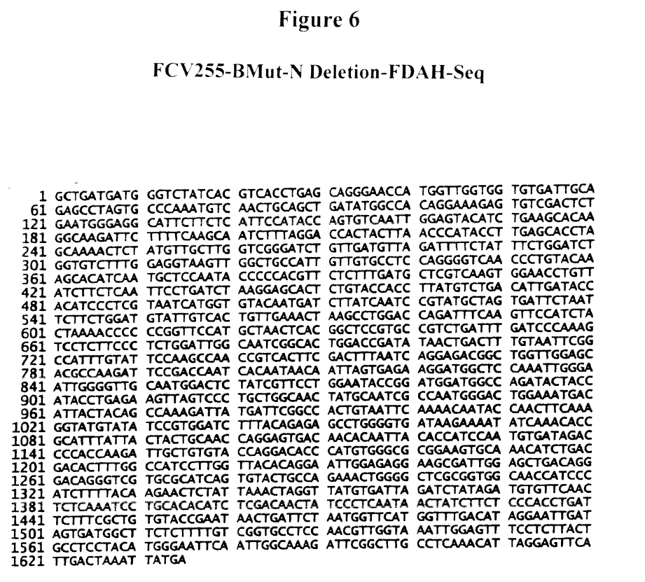

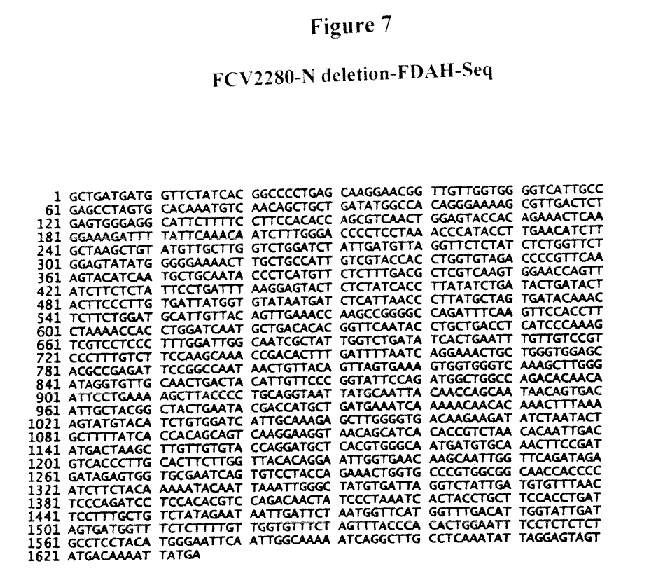

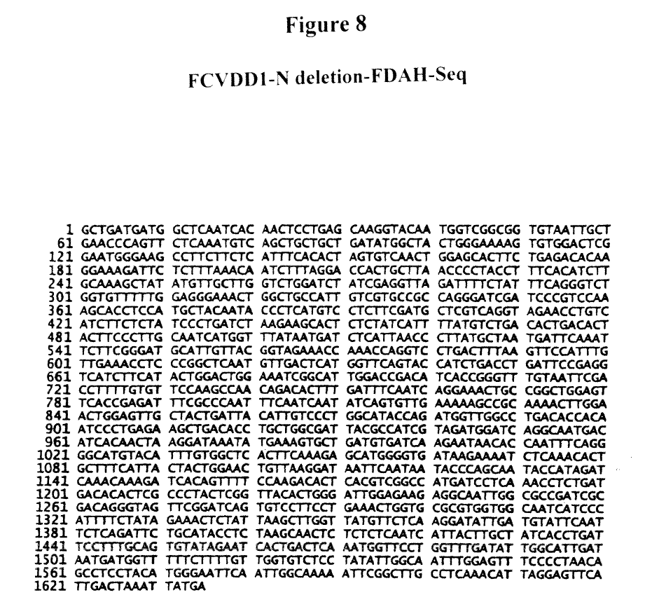

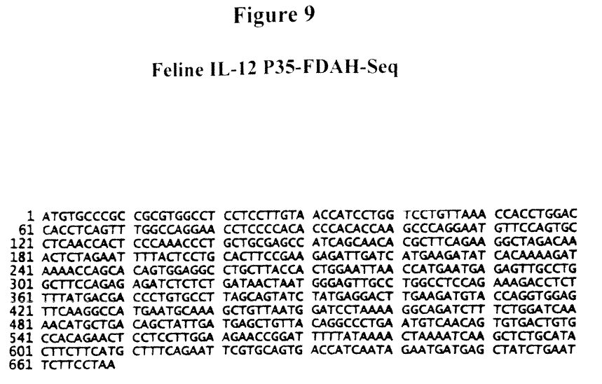

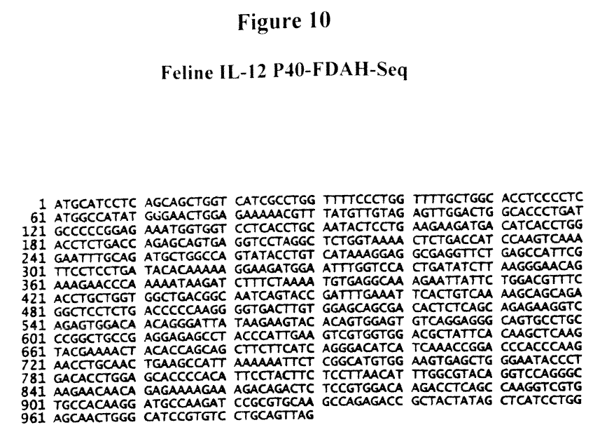

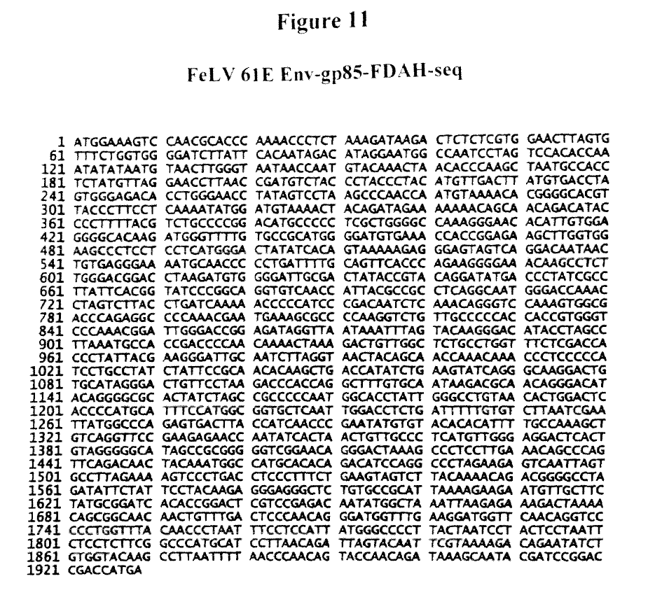

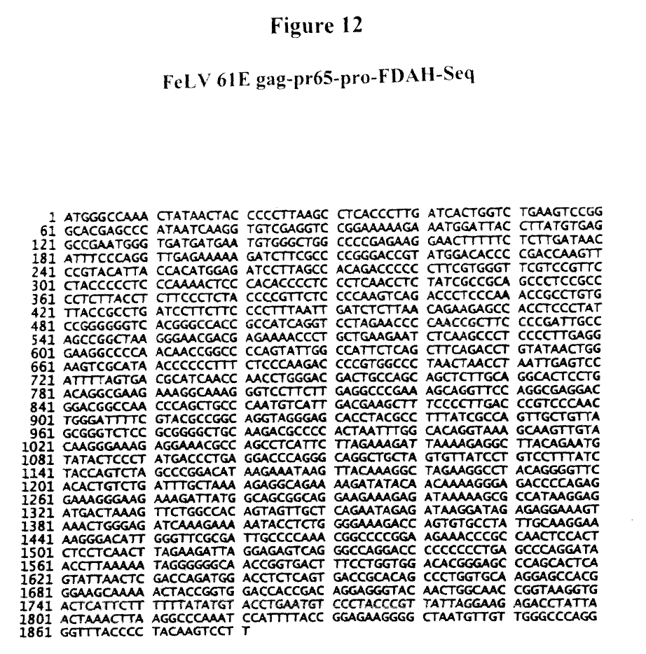

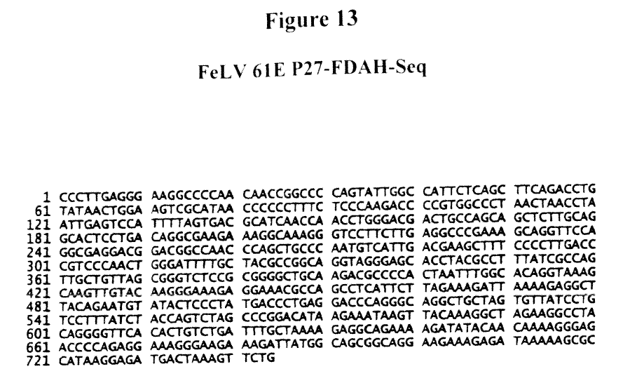

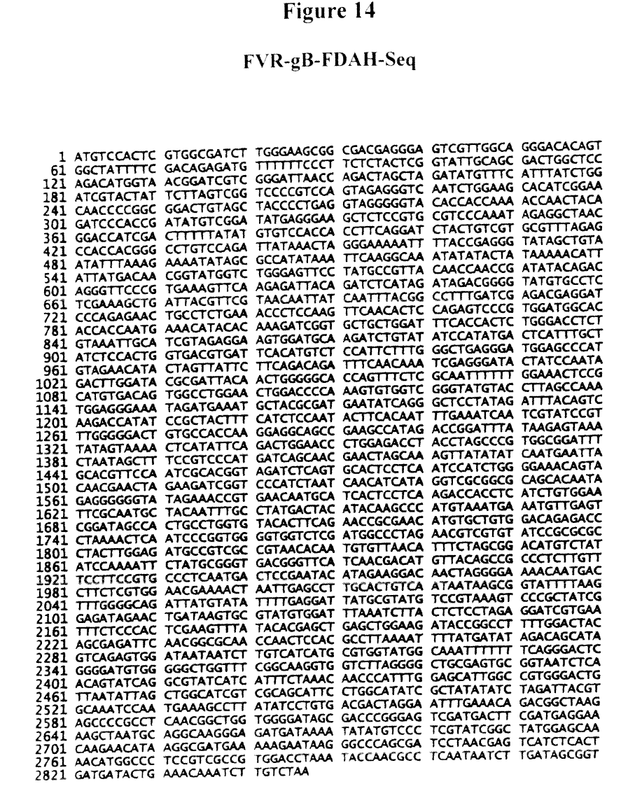

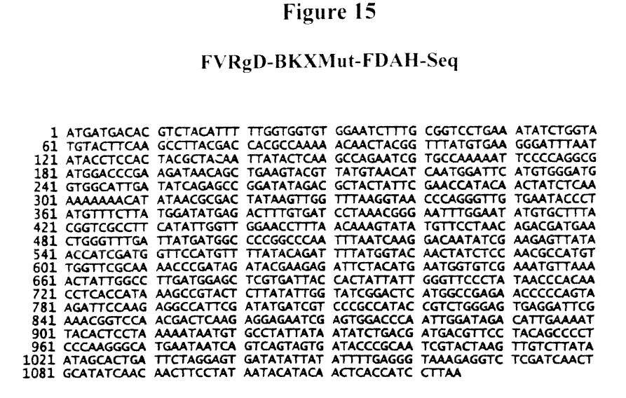

### DETAILED DESCRIPTION OF THE INVENTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Accordingly, in the present application, there may be employed conventional molecular biology, microbiology, and recombinant DNA techniques within the skill of the art. Such techniques are explained fully in the literature. See, e.g., Byrd, C M and Hruby, D E, Methods in Molecular Biology, Vol. 269: Vaccinia Virus and Poxvirology, Chapter 3, pages 31-40; Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Although any methods and materials similar or equivalent to those described herein can be used in the practice or testing of the invention, the preferred methods and materials are now described. All publications mentioned herein are incorporated herein by reference in their entirety.

### DEFINITIONS

The terms used herein have the meanings recognized and known to those of skill in the art, however, for convenience and completeness, particular terms and their meanings are set forth below.

The term “about” means within 20%, more preferably within 10% and more preferably within 5%.

The term “antigen” refers to a compound, composition, or immunogenic substance that can stimulate the production of antibodies or a T-cell response in an animal, including compositions that are injected or absorbed into an animal. The term may be used to refer to an individual macromolecule or to a homogeneous or heterogeneous population of antigenic macromolecules. An antigen reacts with the products of specific humoral or cellular immunity. The term “antigen” broadly encompasses moieties including proteins, polypeptides, antigenic protein fragments, nucleic acids, oligosaccharides, polysaccharides, organic or inorganic chemicals or compositions, and the like. Furthermore, the antigen can be derived or obtained from any virus, bacterium, parasite, protozoan, or fungus, and can be a whole organism. The term “antigen” includes all related antigenic epitopes. Similarly, an oligonucleotide or polynucleotide, which expresses an antigen, such as in nucleic acid immunization applications, is also included in the definition. Synthetic antigens are also included, for example, polyepitopes, flanking epitopes, and other recombinant or synthetically derived antigens (Bergmann et al. (1993) Eur. J. Immunol. 23:2777 2781; Bergmann et al. (1996) J. Immunol. 157:3242 3249; Suhrbier, A. (1997) Immunol. and Cell Biol. 75:402 408; Gardner et al. (1998) 12th World AIDS Conference, Geneva, Switzerland, Jun. 28 Jul. 3, 1998).

“Encoded by” or “encoding” refers to a nucleic acid sequence which codes for a polypeptide sequence, wherein the polypeptide sequence contains an amino acid sequence of at least 3 to 5 amino acids, more preferably at least 8 to 10 amino acids, and even more preferably at least 15 to 20 amino acids, a polypeptide encoded by the nucleic acid sequences. Also encompassed are polypeptide sequences, which are immunologically identifiable with a polypeptide encoded by the sequence. Thus, an antigen “polypeptide,” “protein,” or “amino acid” sequence may have at least 70% similarity, preferably at least about 80% similarity, more preferably about 90-95% similarity, and most preferably about 99% similarity, to a polypeptide or amino acid sequence of an antigen.

The term “exogenous” refers to a foreign gene or protein encoded by such foreign gene that is produced, originated, derived or developed outside the raccoon poxvirus genome.

A “gene” as used in the context of the present invention is a sequence of nucleotides in a nucleic acid molecule (chromosome, plasmid, etc.) with which a genetic function is associated. A gene is a hereditary unit, for example of an organism, comprising a polynucleotide sequence (e.g., a DNA sequence for mammals) that occupies a specific physical location (a “gene locus” or “genetic locus”) within the genome of an organism. A gene can encode an expressed product, such as a polypeptide or a polynucleotide (e.g., tRNA). Alternatively, a gene may define a genomic location for a particular event/function, such as the binding of proteins and/or nucleic acids (e.g., phage attachment sites), wherein the gene does not encode an expressed product. Typically, a gene includes coding sequences, such as polypeptide encoding sequences, and non-coding sequences, such as promoter sequences, poly-adenlyation sequences, transcriptional regulatory sequences (e.g., enhancer sequences). Many eucaryotic genes have “exons” (coding sequences) interrupted by “introns” (non-coding sequences). In certain cases, a gene may share sequences with another gene(s) (e.g., overlapping genes).

An “immune response” to an antigen or vaccine composition is the development in a subject of a humoral and/or a cell-mediated immune response to molecules present in the antigen or vaccine composition of interest. For purposes of the present invention, a “humoral immune response” is an antibody-mediated immune response and involves the generation of antibodies with affinity for the antigen/vaccine of the invention, while a “cell-mediated immune response” is one mediated by T-lymphocytes and/or other white blood cells. A “cell-mediated immune response” is elicited by the presentation of antigenic epitopes in association with Class I or Class II molecules of the major histocompatibility complex (MHC). This activates antigen-specific CD4+ T helper cells or CD8+ cytotoxic T lymphocyte cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cell-mediated immune response” also refers to the production of cytokines, chemokines and other such molecules produced by activated T-cells and/or other white blood cells, including those derived from CD4+ and CD8+T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to restimulation with antigen. Such assays are well known in the art. See, e.g., Erickson et al., J. Immunol. (1993) 151:4189-4199; Doe et al., Eur. J. Immunol. (1994) 24:2369-2376.

An “immunologically effective amount” or an “effective immunizing amount”, used interchangeably herein, refers to the amount of antigen or vaccine sufficient to elicit an immune response, either a cellular (T cell) or humoral (B cell or antibody) response, as measured by standard assays known to one skilled in the art. In the present invention, an “immunologically effective amount” or an “effective immunizing amount” is the minimal protection dose (titer) of about 4.5 to 7.5 Log₁₀TCID₅₀/mL. The effectiveness of an antigen as an immunogen, can be measured either by proliferation assays, by cytolytic assays, such as chromium release assays to measure the ability of a T cell to lyse its specific target cell, or by measuring the levels of B cell activity by measuring the levels of circulating antibodies specific for the antigen in serum. Furthermore, the level of protection of the immune response may be measured by challenging the immunized host with the antigen that has been injected. For example, if the antigen to which an immune response is desired is a virus or a tumor cell, the level of protection induced by the “immunologically effective amount” of the antigen is measured by detecting the percent survival or the percent mortality after virus or tumor cell challenge of the animals.

As defined herein “a non-essential site” in the raccoon poxvirus genome means a region in the viral genome, which is not necessary for viral infection or replication. Examples of non-essential sites in the raccoon poxvirus genome include, but are not limited to, the thymidine kinase (TK) site, the hemagglutinin (HA) site and the serine protease inhibitor site. The TK site of raccoon poxvirus is described in C. Lutze-Wallace, M. Sidhu and A, Kappeler, Virus Genes 10 (1995), pp. 81-84. The sequence of the TK gene of raccoon poxvirus can also be found in PubMed accession numbers DQ066544 and U08228. The HA site of raccoon poxvirus is described in Cavallaro K F and Esposito, J J, Virology (1992), 190(1): 434-9. The sequence of the HA gene of raccoon poxvirus can also be found in PubMed accession number AF375116.

The term “nucleic acid molecule” or “nucleic acid sequence” has its plain meaning to refer to long chains of repeating nucleotides such as the repeated units of purine and pyrimidine bases that direct the course of protein synthesis, that is, they encode and express the protein substance. As the term is used in the claims, the nucleic acid refers to the known exogenous or foreign genes that encode the feline antigens.

“Operably linked” refers to an arrangement of elements wherein the components so described are configured so as to perform their usual function. Thus, a given promoter that is operably linked to a coding sequence (e.g., a sequence encoding an antigen or interest) is capable of effecting the expression of the coding sequence when the regulatory proteins and proper enzymes are present. In some instances, certain control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence. Thus, a coding sequence is “operably linked” to a transcriptional and translational control sequence in a cell when RNA polymerase transcribes the coding sequence into mRNA, which is then trans-RNA spliced and translated into the protein encoded by the coding sequence.

A “protective” immune response refers to the ability of a vaccine to elicit an immune response, either humoral or cell mediated, which serves to protect the mammal from an infection. The protection provided need not be absolute, i.e., the infection need not be totally prevented or eradicated, if there is a statistically significant improvement compared with a control population of feline mammals. Protection may be limited to mitigating the severity or rapidity of onset of symptoms of the infection.

The term “recombinant” as used herein simply refers to the raccoon poxvirus constructs that are produced by standard genetic engineering methods.

The term “replicable” refers to a microorganism, in particular, a virus such as the raccoon poxvirus, that is capable of replicating, duplicating or reproducing in a suitable host cell.

The terms “vaccine” or “vaccine composition” are used interchangeably herein and refer to a pharmaceutical composition comprising at least one immunologically active component that induces an immune response in an animal, and/or protects the animal from disease or possible death due to an infection, and may or may not include one or more additional components that enhance the immunological activity of the active component. A vaccine may additionally comprise further components typical to pharmaceutical compositions.

A “vector” is a DNA molecule, capable of replication in a host organism, into which a gene is inserted to construct a recombinant DNA molecule.

### General Description

In accord with the present invention, there is provided a unique, safe and effective recombinant feline combination (referred to as comb, combination, or multivalent) vaccine using raccoon poxviruses as vectors for expressing multiple feline viral, bacterial and cytokine antigens at the hemagglutinin (ha) and/or the thymidine kinase (tk) insertion loci of the raccoon poxvirus genome. Desirably, the constructs express the nucleic acid molecules (genes) encoding the feline calicivirus (FCV) capsid protein, feline viral rhinotracheitis virus (FVR) glycoproteins D/B (gD/gB), feline Chlamydia psittaci (FCP, now commonly known as Chlamydophila felis) outer membrane protein (momp), feline leukemia virus (FeLV) gag-pr65-pro/env-gp70/gp85, and feline interleukin-12 (IL-12) P351P40, the latter component being included as an immunomodulator to enhance immunogenicity of the comb vaccine in cats without the addition of adjuvants. This new, potent combination vaccine is adjuvant-free and safer in its unique ability to avoid the occasional adjuvant-related sarcoma issues with injection of certain vaccine formulations in cats. Other antigens such as feline panleukopenia virus (FPV, using modified live vaccine strain), feline immunodeficiency virus (FIV), rabies virus, feline infectious peritonitis virus (FIPV), Bartonella bacteria, FCV-Diva, FCV-Kaos, FCV-Bellingham, FCV-F9, FCV-F4, FCV-M8, a combination thereof and the like may be optionally included as additional fractions of the multivalent recombinant vaccine to provide broad spectrum protection in cats to a wide variety of feline pathogenic agents.

Raccoon poxvirus (Herman strain) was first isolated from the respiratory tract of raccoons with no clinical symptoms by Y. F. Herman in Aberdeen, Maryland in 1961-1962 (Y. F. Herman, “Isolation and characterization of a naturally occurring pox virus of raccoons,” In: Bacteriol. Proc., 64th Annual Meeting of the American Society for Microbiology, p. 117 (1964)). Several earlier studies reported that the RCNV vector expressing CVS rabies G gene at the tk locus is safe when administered to both wild animals and domestic animals including cats (see, for example, A. D. Alexander et al. “Survey of wild mammals in a Chesapeake Bay area for selected zoonoses,” J. Wildlife Dis. 8: 119-126 (1972); C. Bahloul et al., “DNA-based immunization for exploring the enlargement of immunological cross reactivity against the lyssaviruses,” Vaccine 16: 417-425 (1998); S. Chakrabarti et al., “Compact, Synthetic, vaccinia virus early/late promoter for protein expression,” BioTechniques 23: 1094-1097 (1997); and J. C. DeMartini et al., “Raccoon poxvirus rabies virus glycoprotein recombinant vaccine in sheep,” Arch. Virol. 133: 211-222 (1993)). Other RCNV constructs containing feline antigens have been previously made for administration to cats as noted herein above and known to those of ordinary skill in the art.

However, none of the earlier constructs provide the unique design of the present invention in which multiple genes encoding the feline antigens are inserted at the hemagglutinin (ha) and/or the thymidine kinase (tk) insertion loci of the raccoon poxvirus genome to provide safe and efficacious activity against a broad variety of feline pathogens.

In contrast to the method of U.S. Pat. No. 5,505,941 in which FeLV env gene containing the sequences which encode the p70+p15E polyprotein is used, the construct of the present invention employs a different and unique combination of proteins drawn to the gag-pr65-pro/env-gp70/gp85 of FeLV. The vector and promoter for generating the rRCNV-FeLV in the new combination vaccine of the present invention are also distinct from the vector and promoter used to make the prior canary poxvirus vector vaccine.

In particular, the rRCNV-FCV fraction of the combination vaccine of the present invention expresses two or more FCV capsid genes. Although desirably, the construct can be made to include and express a single nucleic acid molecule encoding the feline calicivirus capsid protein of FCV-2280 from the hemagglutinin locus of the raccoon poxvirus genome, it is preferable to also insert the gene encoding the FCV-DD1 capsid protein with or without the concomitant insertion of the gene encoding the feline calicivirus capsid protein of FCV-255 into the same hemagglutinin locus.

To isolate the feline calicivirus (FCV) capsid gene useful in the present invention to construct rRCNV-FCV, any strain of feline calicivirus (FCV) may be utilized but preferably at least one of the FCV capsid genes is obtained from the FCV-DD1 strain. This FCV-DD1 strain had been deposited under the conditions mandated by 37 C.F.R. § 1.808 and is being maintained pursuant to the Budapest Treaty in the American Type Culture Collection (ATCC), University Boulevard, Manassas, Va. 20110-2209, U.S.A. Specifically, the FCV-DD1 sample was deposited in the ATCC on Sep. 9, 2004 and assigned ATCC Patent Deposit Designation PTA-6204. The recombinant vaccine fraction may optionally contain the capsid gene of one or more additional FCV isolates such as, for example, FCV-255 (See NCBI/GenBank accession number U07130), FCV-2280 (See NCBI/GenBank accession number X99445), FCV-Diva (See Pedusen, NC, Vet. Microbiol. 73: 281-300 (May 2000); Schorr-Evans, EM, J Feline Med and Surg 5: 217-226 (2003)), FCV-Kaos, FCV-Bellingham, FCV-F9 (See NCBI/GenBank accession number Z11536), FCV-F4 (See NCBI/GenBank accession number D90357), FCV-M8, etc. A particularly preferred construct expresses the antigenic proteins of FCV-2280, FCV-DD1 (See U.S. Pat. No. 7,306,807 and ATCC deposit number PTA-6204) and FCV-255.

It is also found as a unique feature that the rRCNV-FCV constructs can utilize the FCV capsid antigen as a screening marker for cloning purposes and avoid the conventional use of foreign markers such as LacZ.

The rRCNV-FVR gB/gD fraction of the combination vaccine of the present invention is distinctively able to express two protein genes, rRCNV-FVR gD and rRCNV-FVR gB, using the P11 promoter to drive and combine the nucleotide sequences encoding gD and gB into the hemagglutinin locus of the raccoon poxvirus genome. The construct is made by cloning FVR gD (glycoprotein D) into an existing plasmid (pFD2000A FVR gB) to generate the plasmid pFD2000A FVR gB/gD. From there, pool clones are created by three-way infection/transfection of COS7 cells, plasmid pFD2000A FVR gB/gD and rRCNV-FeLV using a blue-to-white screening technique. Clone screening is achieved by limited dilution and a novel use of the antigen FeLV P27 as parent for the clone screening, which avoids the traditional foreign marker LacZ for screening.

The rRCNV-FCP momp fraction of the combination vaccine of the present invention expresses the feline Chlamydia psittaci (FCP, also known as Chlamydophila felis) outer membrane protein (momp) and is constructed using the promoter P11.

The rRCNV-FeLV fraction of the combination vaccine of the present invention expresses the nucleic acid molecules encoding the feline leukemia virus antigens gag-pr65-pro and env-gp85 at the hemagglutinin locus of the raccoon poxvirus genome. Alternatively, the construct can be made to contain and express the genes encoding the feline leukemia virus antigens gag-pr65-pro, env-gp85 and env-gp70 at the thymidine kinase locus of the raccoon poxvirus genome.

The unique rRCNV-Feline IL-12 fraction of the combination vaccine of the present invention expresses feline IL-12 in a same locus (ha or tk) by driving two different expression levels of promoters (P1/P_{SEL} for P35, and P7.5/P_{SEL} for P40) in the same virus. Preferably, the nucleic acid molecules encoding the P35 and P40 antigens of feline interleukin-12 are inserted into the hemagglutinin locus of the raccoon poxvirus genome.

Also, the combination vaccine of this invention may optionally contain other pathogens as antigens in admixture as a simple mixture, suspension, emulsion and the like with the recombinant constructs such as, for example, feline panleukopenia virus, feline immunodeficiency virus, rabies virus, feline infectious peritonitis virus, Baronella bacteria (eg. typical cat scratch disease), a combination thereof and the like. If the capsid gene of a particular feline calicivirus strain is not included within the generated recombinant poxvirus, the viral antigen may be separately added to the multivalent vaccine formulation as an additional fraction such as, for example, FCV-255, FCV-2280, FCV-Diva, FCV-Kaos, FCV-Bellingham, FCV-F9, FCV-F4, FCV-M8, etc.

The present invention additionally provides a new method of protecting felines against infection and disease that comprises administering the potent new, adjuvant-free recombinant vaccines to the cats in need of protection. In the method of the invention, an immunologically effective amount of the vaccines of the present invention is administered to the feline in order to induce a protective immune response to infection or disease caused by a variety of feline pathogens. An effective immunizing amount given to the cat is one in which a sufficient immunological response to the vaccine is attained to protect cats from being infected with the pathogen as required by standard values in the vaccine field. The immunologically effective dosage or the effective immunizing amount that inoculates the cat and elicits satisfactory vaccination effects can be easily determined or readily titrated by routine testing such as, for example, by standard dose titration studies.

The vaccine can be administered in a single dose or in repeated doses, particularly if a booster shot is necessary. Desirably, the vaccine is administered to healthy cats in a single inoculation to provide long term protection.

The vaccine may contain an immunologically effective amount of any one of the recombinant raccoon poxvirus vector constructs described herein. In another particular embodiment, the combination vaccine may contain an immunologically effective amount of any two or more of the recombinant raccoon poxvirus vectored-constructs described herein.

The vaccine can conveniently be administered intranasally, transdermally (i.e., applied on or at the skin surface for systemic absorption), parenterally, orally, etc., or a combination such as oronasal where part of the dose is given orally and part is given into the nostrils. The parenteral route of administration includes, but is not limited to, intramuscularly, subcutaneously, intradermally (i.e., injected or otherwise placed under the skin), intravenously and the like. The intramuscular, subcutaneous and oronasal routes of administration are preferred. Preferably, the vaccine is administered subcutaneously to healthy cats.

The poxvirus vector may be live or inactivated by conventional procedures for preparing 2 inactivated viral vaccines, for example, using BEI (binary ethyleneimine), formalin and the like, with BEI being a preferred inactivant, though it is highly desirable for the vaccine of the present invention to use a live raccoon poxvirus for optimal and potent immunological efficacy. The live raccoon poxvirus is also replicable, meaning it can reproduce in suitable culture to make copies of itself for vaccine development from the master seed virus.

When administered as a liquid, the present vaccine may be prepared in the conventional form of an aqueous solution, syrup, elixir, tincture and the like. Such formulations are known in the art and are typically prepared by dissolution or dispersion of the antigen and other additives in the appropriate carrier or solvent systems for administration to cats. Suitable nontoxic, physiologically acceptable carriers or solvents include, but are not limited to, water, saline, ethylene glycol, glycerol, etc. The vaccine may also be lyophilized or otherwise freeze-dried and then aseptically reconstituted or rehydrated using a suitable diluent shortly before use. Suitable diluents include, but are not limited to, saline, Eagle's minimum essential media and the like. Typical additives or co-formulants are, for example, certified dyes, flavors, sweeteners and one or more antimicrobial preservatives such as thimerosal (sodium ethylmercurithiosalicylate), neomycin, polymyxin B, amphotericin B and the like. Such solutions may be stabilized, for example, by addition of partially hydrolyzed gelatin, sorbitol or cell culture medium, and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Liquid formulations also may include suspensions and emulsions that contain suspending or emulsifying agents in combination with other standard co-formulants. These types of liquid formulations may be prepared by conventional methods. Suspensions, for example, may be prepared using a colloid mill. Emulsions, for example, may be prepared using a homogenizer.

Parenteral formulations, designed for injection into body fluid systems, require proper isotonicity and pH buffering to the corresponding levels of feline body fluids. Isotonicity can be appropriated adjusted with sodium chloride and other salts as necessary. At the time of vaccination, the virus is thawed (if frozen) or reconstituted (if lyophilized) with a physiologically-acceptable carrier such as deionized water, saline, phosphate buffered saline, or the like. Suitable solvents, such as propylene glycol, can be used to increase the solubility of the ingredients in the formulation and the stability of liquid preparations.

Any method known to those skilled in the art may be used to prepare the genetic constructs of the present invention. For example, advantage may be taken of particular restriction sites for insertion of any of the desired nucleic acid sequences into the raccoon poxvirus vector using standard methodologies. Alternatively, one may utilize homologous recombination techniques when the insertion of large sequences is desired, or when it is desirable to insert multiple genes, as described herein. In this method, the plasmid sequences flanking the insertion site into which are to be inserted multiple genes, contain sequences which have sufficient homology with sequences present in the raccoon poxvirus genome to mediate recombination. The flanking sequences must be homologous to a region of the raccoon poxvirus that is non-essential for the growth and propagation of the raccoon poxvirus, such as the hemagglutinin locus, or the thymidine kinase locus, or the serine protease inhibitor locus. Although one promoter may be used to drive the expression of two exogenous genes to be recombined, the use of two promoters in an insertion vector, each promoter operably linked to an individual gene will also provide efficient expression.

### EXAMPLES

The following examples demonstrate certain aspects of the present invention. However, it is to be understood that these examples are for illustration only and do not purport to be wholly definitive as to conditions and scope of this invention. It should be appreciated that when typical reaction conditions (e.g., temperature, reaction times, etc.) have been given, the conditions both above and below the specified ranges can also be used, though generally less conveniently. The examples are conducted at room temperature (about 23° C. to about 28° C.) and at atmospheric pressure. All parts and percents referred to herein are on a weight basis and all temperatures are expressed in degrees centigrade unless otherwise specified.

A further understanding of the present invention may be obtained from the examples that follow below. These working examples are intended to illustrate the invention without limiting its scope.

### Example 1

### Construction of Plasmids pFD2000A, pFD2001TK and pFD2003SEL

The two plasmids pFD2000A and pFD2003SEL were constructed as follows to deliver foreign genes into ha locus of raccoon poxvirus genome. The flanking ha sequences are directly cloned/modified from RCNV genome but not from vaccinia virus, to increase the accuracy and frequency of homologous recombination.

Similarly, the plasmid pFD20001TK was constructed to deliver foreign genes into tk locus of raccoon poxvirus genome. The flanking tk sequences are directly cloned/modified from RCNV genome but not from vaccinia virus, to increase the accuracy and frequency of homologous recombination.

### Example 2

### First Generation of rRCNV-FCV Constructs

The rRCNV-FCV2280 Capsid (P₁₁) was constructed and the FCV capsid expression was confirmed by FCV ELISA and Western blot. The construction procedure and recombinant viral construct evaluation in host animals include the following 6 key steps: (1) Clone FCV2280 capsid gene into plasmid vector pFD2000A to generate the plasmid pFD2000A FCV2280 capsid; (2) Three-ways infection/transfection using COS7 cells, plasmid at Step 1 and RCNV to generate the pool clones rRCNV-FCV2280; (3) Pure clone screening by limited dilution and FCV ELISA; (4) Molecular characterization of rRCNV-FCV2280 by PCR, ELISA, and Western blot; (5) Establish the rRCNV-FCV master seed; and (6) The dose titration study of rRCNV-FCV2280 (P₁₁) was done in cats. The challenge study results indicated that cats vaccinated with rRCNV-FCV2280 at even 7.5 Log₁₀TCID₅₀/mL, showed no significant protection against FCV255 challenge.

In addition, the rRCNV-FCV2280 Capsid (P_{SEL}) was constructed in similar approach as above and the FCV capsid expression was confirmed by FCV ELISA and Western blot.

### Example 3

### Second Generation of rRCNV-FCV Construct

The second generation of rRCNV-FCV was constructed as Example 2 but both FCV2280 and FCV DD1 capsid genes (5′-372 bp nucleotides deletion) were inserted at the ha locus, and the FCV capsid expression was confirmed by FCV ELISA and Western blot. In this construct, recombinant raccoon poxvirus expressed both FCV2280 capsid (P₁₁) and FCV DD1 (P_{SEL}) at the ha locus. The master seed was designated rRCNV-FCV (2280-DD1). The dose titration study was conducted in cats, and the results were summarized as follows: (1) Significant serum neutralization to FCVDD1 titers were observed in 10 cats vaccinated with 7.5 Log₁₀TCID₅₀/mL while all controls (10 cats) remained sero-negative (p<0.05); (2) Significant reduction of fever in vaccinated groups (6.5 and 7.5 Log₁₀TCID₅₀/mL) was observed compared to the controls (p<0.05); (3) Significant reduction of oral and external ulcers (lesions) in the vaccinated group (7.5 Log, TCID₅₀/mL) was observed compared to the control group (p<0.05). These results indicated that rRCNV-FCV (2280-DD1) is useful as a vaccine candidate.

### Example 4

### Third Generation of rRCNV-FCV Construct

In this construct, the FCV capsid genes (2280-DD1-255) is inserted at ha locus. The third generation of rRCNV-FCV was constructed through the following four key steps: (1) Clone FCV255 capsid into existing plasmid (used to construct the 2^{nd} generation construct) to generate the plasmid pFD2000A FCV capsids (2280-DD1-255); (2) Create a pool clones by three-way infection/transfection: COS7 cells, plasmid at Step 1 and RCNV; (3) Clone screening by limited dilution and FCV ELISA; and (4) The insertion of FCV capsid genes into RCNV genome and the expression of FCV capsids was determined by FCV PCR, ELISA and Western blot. The dose titration study is being conducted in cats. This construct will increase the vaccine efficacy and broaden the protection spectrum compared to the second generation construct.

### Example 5

### First Generation of rRCNV-FVR Construct

The rRCNV-FVR gD ((P₁₁) and rRCNV-FVR gB ((P₁₁) were constructed in a similar approach as described in Example 2. The construction procedure includes the following 5 key steps: (1) Clone FVR gD/gB glycoprotein genes into plasmid vector pFD2000A, respectively to generate the plasmids pFD2000A FVR gD, and pFD2000A FVR gB; (2) Three-ways infection/transfection using COS7 cells, plasmid at Step 1 and RCNV to generate the pool clones rRCNV-FVR gD/gB; (3) Pure clone screening by plaque purification/LacZ screening; (4) Molecular characterization of rRCNV-FVR gD (P₁₁) and rRCNV-FVR gB ((P₁₁) by PCR, ELISA, and Western blot; (5) Establish the rRCNV-FVR master seed.

### Example 6

### Second Generation of rRCNV-FVR Construct

The second generation of rRCNV-FVR was constructed through the following four key steps: (1) Clone FVR gD (glycoprotein D) into existing plasmid (pFD2000A FVR gB) to generate the plasmid pFD2000A FVR gB/gD; (2) Create a pool clones by three-way infection/transfection: COS7 cells, plasmid at Step 1 and rRCNV-FeLV (blue-to-white screening, see example 13); (3) Clone screening by limited dilution and FeLV P27 ELISA. In this construct, the FVR gD/gB genes is inserted at ha locus; and (4) The insertion of FVR gD/gB genes into RCNV genome and the expression of FVR gD/gB is determined by FVR PCR and Western blot. The dose titration study is being conducted in cats.

### Example 7

### First Generation of rRCNV-FeLV Construct

The rRCNV-FeLV gag-p 5 ((P₁₁) and rRCNV-FeLV env-gp70 ((P₁₁) were constructed in a similar approach as described in Example 2. The dose titration study of these two constructs indicated that rRCNV-FeLV env-gp70 construct showed 40% prevention (2/5 cats vaccinated with 7.5 Log₁₀TCID₅₀/mL) against FeLV viremia, however, rRCNV-FeLV gag-pr65 showed no protection (0/5 vaccinated cats) against FeLV viremia.

### Example 8

### Second Generation of rRCNV-FeLV Constructs

The second generation of rRCNV-FeLV was constructed. In this construct, recombinant raccoon poxvirus expressed both FeLV gag-pr65-pro (P_{SEL}) and FeLV env-gp85 (P_{SEL}) at the ha locus. The construction procedure and vaccine candidate evaluation in cats include the following 6 key steps: (1) Clone FeLV gag-pr65-pro, and FeLV env gp85 into plasmid vector pFD2003SEL; (2) Construct the plasmid pFD2003SEL FeLV gag-pr65-pro (P_{SEL})-env-gp85 (P_{SEL}); (3) Three-ways infection/transfection using COS7 cells, plasmid at Step 2 and rRCNV-FCV to generate the pool clones rRCNV-FeLV; (3) Pure clone screening by limited dilution and FeLV P27 ELISA; (4) Molecular characterization of rRCNV-FeLV by PCR, ELISA, and Western blot; (5) Establish the rRCNV-FeLV master seed; and (6) The dose titration study in cats. The challenge results were summarized: 7/10 (70%), 6/10 (60%), and 5/10 (50%) cats were protected against persistent FeLV viremia when cats were vaccinated subcutaneously with 7.5, 6.5 and 5.5 Log₁₀TCID₅₀/mL rRCNV-FeLV, respectively, in a two-dosage regimen (3-weeks interval). By contrast, 9/10 (90%) non-vaccinated cats showed persistent FeLV viremia. In view of the failure of the earlier first generation constructs, these unexpectedly successful results indicated that rRCNV-FeLV is useful as a vaccine candidate.

### Example 9

### Third Generation of rRCNV-FeLV Construct

In this construct, the FeLV gag-pr65-pro/env-gp85 genes is inserted at tk locus. The third generation of rRCNV-FeLV was constructed through the following four key steps: (1) Generate the plasmid pFD2006TK FeLV gag-pr65/env-gp85; (2) Create a pool clones by three-way infection/transfection: COS7 cells, plasmid at Step 1 and rRCNV-FeLV env-gp70 (P₁₁) (from the first generation construct, Example 7); (3) Clone screening by limited dilution and FeLV P27 Elisa; and (4) The insertion of FeLV gag/env genes into RCNV genome and the expression of FeLV gag/env is determined by FeLV P27 ELISA and FeLV gp70 Western blot. The dose titration study is being conducted in cats.

### Example 10

### First Generation of rRCNV-FCP Construct

The rRCNV-FCP outer membrane protein (momp, P₁₁) was constructed (ha locus) in a similar approach as described in Example 2. The construction procedure includes the following key steps: (1) Clone FCP momp gene into plasmid vector pFD2000A to generate the plasmid pFD2000A FCPmomp (P₁₁); (2) Three-ways infection/transfection using COS7 cells, plasmid at Step 1 and RCNV to generate the pool clones rRCNV-FCP; (3) Pure clone screening by plaque purification/LacZ screening; (4) Molecular characterization of rRCNV-FCP momp (P₁₁) by PCR; and (5) Establish the rRCNV-FCP momp master seed.

### Example 11

### First Generation of rRCNV-Feline IL-12 Construct

The feline IL-12 P35 and P40 genes were cloned from the lymph node tissue of cats by RT-PCR and TOPO cloning, and the feline IL-12 P35 and P40 genes were sequenced. The rRCNV-Feline IL-12 P35 (P11) and rRCNV-Feline IL-12 (P11) was constructed as Example 2. Feline IL-12 P35 and P40 expression at ha locus was determined by P40-specific Western blot.

### Example 12

### Second Generation of rRCNV-Feline IL-12 Construct

In this construct, the feline IL-12 P35/P40 genes are inserted at ha locus. The second generation of rRCNV-FeLV IL-12 is constructed through the following four key steps: (1) Construct the plasmid pFD2003SEL Feline IL-12 P35/P40; (2) Create a pool clones by three-way infection/transfection: COS7 cells, plasmid at Step 1 and rRCNV-FeLV (blue-to-white screening or feline IL-12 P40 ELISA); (3) Clone screening by limited dilution and FeLV P27 Elisa or Feline IL-12 P40 ELISA; and (4) The insertion of feline IL-12 P35/P40 genes into RCNV genome and the expression of feline IL-12 P35/P40 is determined by feline 11-12 PCR and P40 Western blot. The dose titration study (respective feline antigen formulated with live or inactivated rRCNV-feline IL-12) is being conducted in the cats to evaluate the enhancing effect of feline IL-12 cytokine on immunity.

### Example 13

### Blue-To-White Screening Marker for Recombinant RCNV Vector System

The rRCNV-FeLV gag (first generation construct), and rRCNV-FeLV (gag-pr65-pro//env gp85, second generation construct) is used as parent for blue-to-white (btw) screening to construct any RCNV-vectored recombinant vaccine. The beauty of this system is that it takes advantage of rRCNV-FeLV as parent strain (blue plaque due to FeLV P27 gene expression) rather than RCNV wild type (white plaque), and any foreign interest gene (or protective antigen) is inserted at ha/tk locus to replace FeLV P27 containing DNA fragment flanking within ha/tk DNA sequence by allelic exchange. Consequently, FeLV P27 antigen ELISA can easily differentiate the recombinant (white plaque) from parent strain (blue plaque due to FeL P27 expression) in 96-well plate screening system. This system is used to construct rRCNV-Feline IL-12 and rRCNV-FVR.

### Example 14

### Expression of Inserted Immuogen as Screening Marker in Recombinant RCNV-Vector System

This concept was applied in the construction of rRCNV-FCV (see above Examples 3 and 4). The resulting recombinant clones were screened by FCV capsid-specific ELISA. The unique feature is that no foreign marker such as LacZ is required for screening. This concept can be used in all rRCNV-viral constructs of the present invention only if the immunogen-specific ELISA is available, for example, rRCNV-FeLV (P27 ELISA), rRCNV-feline IL-12 (P40 ELISA), etc.

### Example 15

### Virus Stability

Survivability of the microorganism in the field environment and laboratory conditions was tested. Under the laboratory conditions, the construct, rRCNV FIPV-N (recombinant RCNV expressing feline infectious peritonitis virus nucleocapsid gene), has been tested by holding the virus stocks prepared at the highest passage (MSV+5) at −70, 4-8, and 37° C. Samples of the virus stocks were removed at specified intervals and titrations were performed to determine the stability of this virus under various storage conditions. Both lyophilized cakes of virus containing stabilizer and liquid suspensions of the virus were found to be stable, as indicated by no significant loss of virus titer, when stored at −70° C. and 4-8° C. for 90 days, and storage under 4-8° C. for 33 months. At 37° C. a significant reduction in virus titer was observed on day 14 for the liquid virus. The virus was no longer detectable by this assay by day 28 at 37° C. This virus seems to be quite stable when stored refrigerated.

In the foregoing, there has been provided a detailed description of particular embodiments of the present invention for purpose of illustration and not limitation. It is to be understood that all other modifications, ramifications and equivalents obvious to those having skill in the art based on this disclosure are intended to be included within the scope of the invention as claimed.

### Example 16

### Second Generation of rRCNV-Feline IL-12 Construct

Briefly, the virus rRCNV-Feline IL-12 was constructed by insertion of the feline IL-12 P35 and P40 genes into the hemagglutination (ha) locus of the RCNV genome, an avirulent Herman strain. The feline IL-12 P35 and P40 genes were cloned from cat lymphoid node using RT-PCR.

The construction processes of rRCNV-Rabies G2 were provided through two major steps. First, the PCR-amplified 669-bp P35 and 990-bp P40 genes of feline IL-12 was subcloned into a plasmid pFD2003SEL vector to generate plasmid pFD2003SEL-Feline IL-12 P35-(SEL)-P40. Both P35 and P40 genes are co-expressed under the control of promoter P_{SEL}, respectively. Second, three-way co-infection/transfection of RCNV and plasmid pFD2003SEL-Feline IL-12 (P35-P40) in COS-7 cells was conducted to generate rRCNV-Feline IL-12 by allelic exchange at the ha locus. The feline IL-12-expressed clones were screened by four successive rounds of limited dilutions and P40 ELISA in Vero cells. The clone candidates were further expanded two more times in Vero cells using Minimum Essential Medium (MEM) supplemented with 0.05% lactalbumin hydrolysate (LAH), 30 μg/mL gentamicin sulfate and 5% fetal bovine serum and thereafter confirmed by feline IL-12 P40 gene-specific PCR and feline IL-12 P40 ELISA. The sixth passage was used to prepare a pre-master seed. The Master Seed was established by a 1:10,000 dilution of pre-master seed, and designated rRCNV-Feline IL-12, in which the raccoon poxvirus as a live vector is capable of expressing the feline IL-12 P35 and P40 proteins, respectively, at the ha loci. The dose titration study using rRCNV-FPV/FCV/FVR/FCP/FeLV/Rabies and different doses of rRCNV-feline IL-12 is being conducted in the cats to evaluate the enhancing effect of feline IL-12 cytokine on immunity.

What is claimed is:

1. A recombinant raccoon poxvirus vector (rRCNV) comprising two or more exogenous, homologous nucleic acid molecules, each encoding a protein from two or more different strains of the same feline pathogen, wherein at least two of the nucleic acid molecules are inserted into the hemagglutinin (ha) locus or the thymidine kinase (tk) locus, or at least one of the nucleic acid molecules is inserted into each of the hemagglutinin and thymidine kinase loci.

2. The recombinant raccoon poxvirus vector according to claim 1, wherein the raccoon poxvirus is live and replicable.

3. The recombinant raccoon poxvirus vector according to claim 1, wherein at least one of the exogenous nucleic acid molecules encodes a feline calicivirus capsid protein.

4. The recombinant raccoon poxvirus vector according to claim 1, wherein at least one of the exogenous nucleic acid molecules encodes the feline calicivirus capsid protein of FCV-2280.

5. The recombinant raccoon poxvirus vector according to claim 3, wherein the nucleic acid molecule that encodes the FCV-2280 capsid protein is operably linked to a early-late promoter for expression, which promoter may be a synthetic early-late promoter for expression.

6. The recombinant raccoon poxvirus vector according to claim 1, further comprising a nucleic acid molecule encoding the feline calicivirus capsid protein of FCV-DD1 inserted into the hemagglutinin locus or the thymidine kinase locus of the raccoon poxvirus genome

7. The recombinant raccoon poxvirus vector according to claim 5, wherein the nucleic acid molecule that encodes the FCV-2280 capsid protein is operably linked to a vaccinia virus late promoter for expression and the nucleic acid molecule that encodes the FCV-DD1 capsid protein is operably linked to an early-late promoter for expression, which promoter may be a synthetic early-late promoter for expression.

8. The recombinant raccoon poxvirus vector according to any of claims 1-7, further comprising a nucleic acid molecule encoding the feline calicivirus capsid protein of FCV-255 inserted into the hemagglutinin locus or the thymidine kinase locus of the raccoon poxvirus genome.

9. The recombinant raccoon poxvirus vector according to claim 1, further comprising at least one exogenous nucleic acid molecule that encodes the feline viral rhinotracheitis virus glycoprotein gD or the feline viral rhinotracheitis virus glycoprotein gB.

10. The recombinant raccoon poxvirus vector according to claim 1, further comprising at least one exogenous nucleic acid molecule that encodes a feline leukemia virus protein.

11. The recombinant raccoon poxvirus vector according to claim 10, further comprising at least one exogenous nucleic acid molecule that encodes at least one of a feline leukemia virus env protein or a gag protein.

12. The recombinant raccoon poxvirus vector according to claim 10, wherein the nucleic acid molecules encoding the viral antigens are operably linked to an early-late promoter for expression, which promoter may be a synthetic early-late promoter for expression.

13. The recombinant raccoon poxvirus vector according to claim 1, further comprising at least one heterologous nucleic acid molecule encoding a feline protein from a different feline pathogen.

14. The recombinant raccoon poxvirus vector according to either of claims 1 or 13, further comprising at least one exogenous nucleic acid molecule that encodes a Chlamydophila felis protein.

15. The recombinant raccoon poxvirus vector according to claim 1, further comprising at least one exogenous nucleic acid molecule that encodes an outer membrane protein of Chlamydophila felis.

16. The recombinant raccoon poxvirus vector according to claim 15, wherein the nucleic acid sequence of the outer membrane protein gene is operably linked to a vaccinia virus late promoter for expression.

17. The recombinant raccoon poxvirus vector according to claim 1, further comprising a nucleic acid molecule encoding the P35 protein of feline interleukin-12 and a nucleic acid molecule encoding the P40 protein of feline interleukin-12, which are inserted into the hemagglutinin locus or the thymidine kinase locus of the raccoon poxvirus genome.

18. The recombinant raccoon poxvirus vector according to claim 1, further comprising a nucleic acid molecule encoding a feline protein that is inserted into a third non-essential site of the raccoon poxvirus genome in addition to the thymidine kinase and the hemagglutinin loci of the raccoon poxvirus genome.

19. The recombinant raccoon poxvirus vector of claim 18, wherein the third non-essential site of the raccoon poxvirus genome is the serine protease inhibitor site.

20. A feline vaccine comprising an immunologically effective amount of the recombinant raccoon poxvirus vector of claim 1 and, optionally, a suitable carrier or diluent.

21. A feline vaccine comprising an immunologically effective amount of two or more of the recombinant raccoon poxvirus vectors of any one of claims 1, 3, 13 or 18, and optionally, a suitable carrier or diluent.

22. The feline vaccine according to claim 21 wherein the vaccine further comprises a mixture of one or more additional feline antigens selected from the group consisting of feline panleukopenia virus, feline immunodeficiency virus, rabies virus, feline infectious peritonitis virus, Bartonella bacteria, FCV-Diva, FCV-Kaos, FCV-Bellingham, FCV-F9, FCV-F4, FCV-M8 and a combination thereof.

23. The feline vaccine according to claim 21, wherein the vaccine comprises a mixture of two or more of the recombinant raccoon poxvirus vectors selected from the group consisting of rRCNV-FCV2280, rRCNV-FCV2280-FCVDD1, rRCNV-FCV2280-FCVDD1-FCV255, rRCNV-FVR gD, rRCNV-FVR gB, rRCNV-FVR gD+gB, rRCNV-FeLV gag-pr65-pro/env-gp85, rRCNV-FeLV gag-pr65-pro-env-gp85 (TK)//env-gp70 (HA), rRCNV-FCP momp and rRCNV-feline IL-12 P35/P40.

24. A method for inducing a protective immune response to a feline pathogen in a cat comprising administering to the cat an effective immunizing amount of the vaccine of claim 21.

25. A plasmid comprising the nucleotide sequence of any one of SEQ ID NOs: 1, 2, 3 or 4.

26. The method of claim 24, wherein the effective immunizing amount of the vaccine is at least about 4.5 Log₁₀TCID₅₀/ml.

27. The method of claim 24, wherein the vaccine is administered as a single dose or as repeated doses.

28. The vaccine of claim 1, wherein the vaccine is adjuvant-free.

29. A recombinant raccoon poxvirus vector (RCNV) comprising:
a) at least one exogenous nucleic acid and at least one homologous exogenous nucleic acid each encoding the same feline protein from two or more different strains of a feline pathogen, wherein the at least one homologous nucleic acid is inserted into at least one of either the ha locus, or the tk locus; or
b) at least two exogenous nucleic acids, each nucleic acid encoding at least one different feline protein from the same feline pathogen; wherein one of the exogenous nucleic acids is inserted into at least one of either the ha or the tk site, or, wherein at least one nucleic acid is inserted into the ha locus and at least one nucleic acid is inserted into the tk locus.

30. The recombinant raccoon poxvirus vector (RCNV) of either of claims 1 or 29, comprising at least two homologous, exogenous nucleic acid molecules, each encoding the same feline protein from two or more different strains of a feline pathogen, wherein the homologous, exogenous nucleic acid molecules are inserted into the ha locus, the tk locus, the serine protease inhibitor locus, or wherein at least one homologous exogenous nucleic acid molecule is inserted into each of the ha, tk, or serine protease inhibitor loci.

31. The recombinant raccoon poxvirus vector of claim 29, wherein at least one of the at least two exogenous nucleic acid molecules encodes one different feline protein selected from the group consisting of a feline calicivirus protein, glycoprotein gB of feline rhinotracheitis, glycoprotein gD of feline rhinotracheitis, a gag protein from feline leukemia virus, an env protein from feline leukemia virus, a Chlamydophila felis protein, and a P35 and P40 protein of feline interleukin-12.

32. A feline vaccine comprising an immunologically effective amount of two or more recombinant raccoon poxvirus vectors selected from either one of claims 1 or 29.

33. The feline vaccine according to claim 29, further comprising a mixture of one or more additional feline antigens selected from the group consisting of feline panleukopenia virus, feline immunodeficiency virus, rabies virus, feline infectious peritonitis virus, Bartonella bacteria, FCV-Diva, FCV-Kaos, FCV-Bellingham, FCV-F9, FCV-F4, FCV-M8 and a combination thereof.

34. A method for inducing a protective immune response to a feline pathogen in a cat comprising administering to the cat an effective immunizing amount of the vaccine of claim 33.

35. The method of claim 34, wherein the effective immunizing amount of the vaccine is at least about 4.5 Log₁₀TCID₅₀/ml.
